(19) United States
(12) Patent Application Publication
(10) Pub. No.: US 2024/0408276 A1
(43) Pub. Date: Dec. 12, 2024

(54) AGENTS FOR CARTILAGE RESTORATION AND USES THEREOF

(71) Applicant: University of Connecticut, Farmington, CT (US)

(72) Inventors: Caroline Dealy, Farmington, CT (US); Syam Nukavarapu, Storrs, CT (US)

(21) Appl. No.: 18/737,284

(22) Filed: Jun. 7, 2024

Related U.S. Application Data
(60) Provisional application No. 63/471,777, filed on Jun. 8, 2023.

(51) Int. Cl. A61L 27/36; A61L 27/02; A61L 27/20; A61L 27/22; A61L 27/24; A61L 27/38; A61L 27/52

(52) U.S. Cl. CPC A61L 27/3654; A61L 27/025; A61L 27/20; A61L 27/222; A61L 27/225; A61L 27/227; A61L 27/24; A61L 27/3612; A61L 27/3852; A61L 27/52; A61L 2430/06

(57) ABSTRACT

Disclosed herein are methods for restoring, repairing, or regenerating cartilage in a subject in need of treatment thereof by including at least one natural growth factor in a composition that is injected or implanted into a subject.

### RELATED APPLICATION INFORMATION

The application claims priority to U.S. Application No. 63/471,777, filed on Jun. 8, 2023, the contents of which are herein incorporated by reference.

### FIELD OF THE DISCLOSURE

The present disclosure relates to the use of natural growth factors for cartilage restoration, repair, or regeneration in a subject in need thereof. In some embodiments, the present disclosure relates to the use of natural growth factors for joint cartilage restoration, repair, or regeneration in a subject in need thereof.

### BACKGROUND

Surgical procedures are used to repair focal damage in the cartilage of the joints. Unfortunately, the clinical success of most surgical cartilage repair procedures is poor, with synthetic scaffolds and cartilage in vitro cell-based implants having limited or unproved efficacy and durability and which may only provide a few years of use. The most durable procedure is osteochondral allografting/autografting (OCA) in which a piece of living osteochondral tissue (cartilage+bone) is implanted into the damaged region. However, even OCA is not a permanent solution as it only lasts 5-10 years.

The reason that surgical cartilage repair procedures do not work well relates to the biology of cartilage itself. Joint cartilage has essentially no regenerative capacity, and injuries including surgical incision are followed by fibrocartilage or scar-like tissue formation, not durable replacement cartilage. An agent is needed that can stimulate joint cartilage regenerative potential and healing.

### SUMMARY

In one embodiment, disclosed herein is a method for restoring, repairing, or regenerating cartilage in a mammal in need thereof. In some embodiments, the method comprises the step of: injecting or implanting in to a joint of a mammal a composition, where the composition comprises at least one of (a) a tissue (such as, for example, an osteochondral allo/autograft); (b) a natural or synthetic scaffold, or combination thereof; (c) a hydrogel; (d) a membrane; (e) chondrocytes or chondrogenic cells; (f) a particle; or (g) any combination of (a)-(f), and further wherein any of (a)-(g) of the composition have been treated with or exposed to at least one natural growth factor prior to injection or implantation into the joint of the mammal. In some embodiments, the method is for use in restoring, repairing, or regenerating joint cartilage.

In some embodiments of the above method, the natural growth factor is heparan-binding epidermal growth factor (HB-EGF), epidermal growth factor (EGF), transforming growth factor alpha (TGF-α), amphiregulin (AREG), betacellulin (BTC), epiregulin (EREG), epigen, neuregulin 1-4 (NRG1-4) and isoforms thereof, or a bone morphogenetic protein. In some embodiments of the above method, the natural growth factor is HB-EGF. In yet other embodiments of the above method, the at least one natural growth factor is a bone morphogenetic protein (BMP). In some embodiments, the at least one natural growth factor is BMP2, BMP3, BMP3B, BMP4, BMP5, BMP6, BMP7, BMP8, BMP8B, BMP9, BMP10, BMP12, BMP13, BMP14, BMP15 or any combination thereof. In yet further embodiments of the above method, the natural growth factor is a combination of HB-EGF and at least one BMP.

In still further embodiments of the above method, the mammal is a human, pig, dog, cat, cow, horse, mouse, guinea pig, goat, sheep, or horse. In further embodiments, the mammal suffers from Osteochondritis Dissecans. In still further embodiments, the mammal suffers from osteoarthritis.

In still further embodiments of the above method, the composition comprises a natural or synthetic scaffold or combination thereof and further wherein the (a) natural scaffold comprises, collagen, aragonite, gelatin, elastin, hyaluronic acid, chitosan, chondroitin sulfate, agarose, alginate, cellulose, fibrin, or any combinations; thereof; and (b) synthetic scaffold comprises poly(ethylene glycol) (PEG), polycaprolactone (PCL), polylactic acid (PLA), polyurethane (PU), poly(glycolic acid) (PGA), polyethersulfone (PES), poly-p-dioxanone (PDS), poly(l-lactide) (PLLA), polylactic-co-glycolic acid (PLGA), polycaprolactone-polyethylene glycol (PCEC), polysulfone, or any combinations thereof.

In still other embodiments of the above method, the composition comprises tissue and the tissue is osteochondral tissue, chondral tissue, or a combination thereof. In other embodiments, the osteochondral tissue, chondral tissue, or combination thereof is an allograft or an autograft.

In still further embodiments of the above method, the composition comprises a particle and the particle is a microsphere.

In still further embodiments of the above method, the joint is a knee, a hip, a shoulder, an elbow, a wrist, an ankle, a digit, a spine, a temporomandibular joint, or any combination thereof.

In yet other embodiments of the above method, the composition further comprises heparanse, heparan sulfate, or a combination thereof.

In another embodiment, the present disclosure relates to a method of increasing the shelf-life of a composition comprising an osteochondral or chondral allograft or osteochondral or chondral autograft. In some embodiments, the method comprises adding at least one heparan-Binding Epidermal Growth Factor (HB-EGF), a bone morphogenetic protein, or a combination thereof, to a composition comprising an osteochondral or chondral allograft or osteochondral or chondral autograft. In another embodiment, the method comprises adding heparanse, heparan sulfate, or a combination thereof to a composition comprising an osteochondral or chondral allograft or autograft. In a further embodiment, the method comprises pretreating with heparanase and/or heparan sulfate before adding HB-EGF or a bone morphogenetic protein, or a combination thereof to the composition.

In some embodiments of the above method, the shelf-life of the composition comprising the HB-EGF and/or heparanase and/or heparan sulfate is increased at least one week when compared to a composition comprising an osteochondral or chondral allograft or osteochondral or chondral autograft that does not contain HB-EGF and/or heparinase and/or heparan sulfate.

In still yet other embodiments of the above method, the method further comprises adding a particle comprising at least one natural growth factor to the composition. In some embodiments, the at least one natural growth factor is HB-EGF, BMP2, BMP9, or any combinations thereof. In still yet other embodiments, the particle is a microsphere. In yet still other embodiments, the HB-EGF is coated on the microsphere or embedded in the microsphere. In some embodiments, the at least one natural growth factor is HB-EGF. In still other embodiments, the at least one natural growth factor is a bone morphogenetic protein. In some embodiments, the at least one natural growth factor is BMP2 and/or BMP9. In yet still other embodiments, the BMP2 and/or BMP9 is coated on the microsphere or embedded in the microsphere.

### BRIEF DESCRIPTION OF THE FIGURES

FIG. 1 shows histological sections of explants of human osteochondral tissue harvested from donor allografts, subjected to full thickness cartilage injury using a dermal punch, and then maintained in vitro for 28 days. Views are sagittal. In the Control explant (A) at top, note that the full thickness injuries (one is boxed) extend from the surface of the tissue all the way through the articular cartilage and into the underlying subchondral bone. The boxed area is shown in high magnification in (A′). Note that even after 28 days, an obvious injury gap persists. In contrast, in explants maintained for 28 days after injury in the presence of HBEGF added to the media (25-500 ng/ml) continuously, the injury gap is considerably thinner and one injury is partially healed together (see box in (B) and high magnification view in (B′). Note also the more intense Safranin-O staining in the HB-EGF treated explant (* in B compared to the similar region in A). Panels (C and D) show medium magnification views of the central portion of the Control explant and the HB-EGF treated explant shown in (A and B). Note that the cartilage layer of the HB-EGF treated explant (D) is thicker than that of the Control explant (C), as determined by the height of the line drawn from the cartilage surface down through the noncalcified and calcified layers of the cartilage and to the top of the underlying subchondral bone (compare black lines in C, D). The graph in (E) shows measurements taken from digital images of HB-EGF treated explants and Control explants, demonstrating that the thickness of full cartilage layer of the HB-EGF treated explants is very significantly greater than Control explants (which did not receive HB-EGF) (n=3 each group).

FIG. 2 shows histological sections of explants of human osteochondral tissue harvested from donor allografts, subjected to full thickness cartilage injuries using a dermal punch as before, and then maintained in vitro for 28 days. Views are sagittal; only one injury is shown. In the Control explant (A) at top, the full thickness injury (boxed) is obvious and extends from the surface of the tissue all the way through the articular cartilage and into the underlying subchondral bone, even after 28 days in vitro. The boxed area is shown in high magnification in (A′). In contrast, in the explant maintained for 28 days after injury in the presence of HBEGF added to the media (25-500 ng/ml) intermittently (B), the injury (boxed area) is partially healed together. Notably, in the high magnification shown in B′, it can be seen that the partial healing of the injury is seamless (white arrow in B′). Note also the greater cellularity in the HB-EGF treated explant (small white arrows in B′) especially in the area of seamless healing. Panels (C and D) show medium magnification views of the central portion of the Control explant and the HB-EGF treated explant shown in (A and B). Note that the cartilage layer of the HB-EGF treated explant (D) is thicker than that of the Control explant (C), as determined by the height of the line drawn from the cartilage surface down through the noncalcified and calcified layers of the cartilage and to the top of the underlying subchondral bone (compare black lines in C, D). Additionally, as shown in the high magnification images in C′ and D′, note that the area of the calcified portion of the cartilage layer (below the tidemark but above the bone, outlined in white line) is reduced in the HB-EGF treated explant compared to the Control explant. Moreover, the thickness of the superficial/surface zone, which does not stain intensely with SafraninO because it lacks abundant aggrecan proteoglycan (indicated by white lines in C and D) is greatly reduced in the HB-EGF treated explant compared to the Control explant. The graphs in (E-G) show measurements taken from digital images of HB-EGF treated explants and Control explants. In (E) note the thickness of the full cartilage layer of the HB-EGF treated explants is very significantly greater than Control explants (which did not receive HB-EGF) (n=3 each group). In (F) note the thickness/area of the calcified zone of the HB-EGF treated explants is significantly reduced compared to Control explants (n=3 each group). In (G) note the depth of the superficial/surface zone which fails to stain with SafraninO is very significantly reduced in HB-EGF treated explants compared to Control explants (n=3 each group).

FIG. 3 shows histological sections of explants of human osteochondral tissue harvested from donor allografts, subjected to full thickness cartilage injuries using a dermal punch as before, and then either pre-treated for 2 days with heparanase (also known as heparitinase) or with mock treatment as control, and then maintained in vitro for 28 days with intermittent HB-EGF treatment. Views are sagittal; only one injury is shown. In the Control explant (A) at top, the full thickness injury (boxed) is obvious and extends from the surface of the tissue all the way through the articular cartilage and into the underlying subchondral bone, even after 28 days in vitro. The boxed area is shown in high magnification in (A′). In contrast, in the explant that was injured and then pre-treated with heparanase for 2 days before being maintained for 28 days in the presence of HBEGF added to the media (25-500 ng/ml) intermittently (B), the injury (boxed area) has healed together from bottom to top. Notably, in the high magnification shown in B′, it can be seen that the complete bottom-to-top healing of the injury is seamless (white arrows in B′). Note also that the cartilage layer of the explant pre-treated with heparanase followed by HB-EGF (B′) is thicker than that of the Control explant (A′), as determined by the height of the line drawn from the cartilage surface down through the noncalcified and calcified layers of the cartilage and to the top of the underlying subchondral bone (compare black lines in A′ and B′). Moreover, the thickness of the superficial/surface zone, which does not stain intensely with SafraninO because it lacks abundant aggrecan proteoglycan (indicated by white lines in A′ and B′) is greatly reduced in the explant that was pre-treated with heparanase and then followed by HB-EGF treatment compared to the Control explant (compare white lines in A′ and B′). The graphs in (C, D) show measurements taken from digital images of mock or heparanase pretreated explants that subsequently received intermittent HBEGF for 28 days. In (C) note the thickness of the full cartilage layer of the heparanase pre-treated HB-EGF treated explants (light gray bar labeled HBEGF) is greater than mock pretreated HB-EGF treated explants (dark gray bar labeled CT) but not significantly so (n=3 each group). In (D) note the depth of the superficial/surface zone which fails to stain with SafraninO is significantly reduced in the heparanase pre-treated HB-EGF treated explants (light gray bar labeled HBEGF) compared to the mock-pretreated HB-EGF treated explants (dark gray bar labeled CT) (n=3 each group).

FIG. 4 shows testing of the mechanical strength of cartilage integrative healing by push out test. The test is diagrammed in (A) illustrating how the end of a piston is positioned on the surface of the central region of the injured explant and force (from a load cell, not illustrated) is applied until the piston pushes the central portion of the explant out the other side. Because the subchondral bone and calcified zone is removed prior to testing, the amount of force needed to push out the center core is a readout of the strength of cartilage integrative healing of the dermal punch injuries. In (B), the force applied is plotted during the test, showing the breakpoint (arrow) when the core is pushed out. The graph in (C) compares pushout force needed to expel the central core from human osteochondral explants immediately after injury (day 0, black bar); or 14 or 42 days after injury (light gray and dark gray bars, respectively) when maintained during that time in control media (no treatment). Surprisingly, there was no significant difference in pushout force needed to expel the core at any time point, consistent with the complete lack of cartilage integrative healing observed histologically in the control human osteochondral explants in vitro.

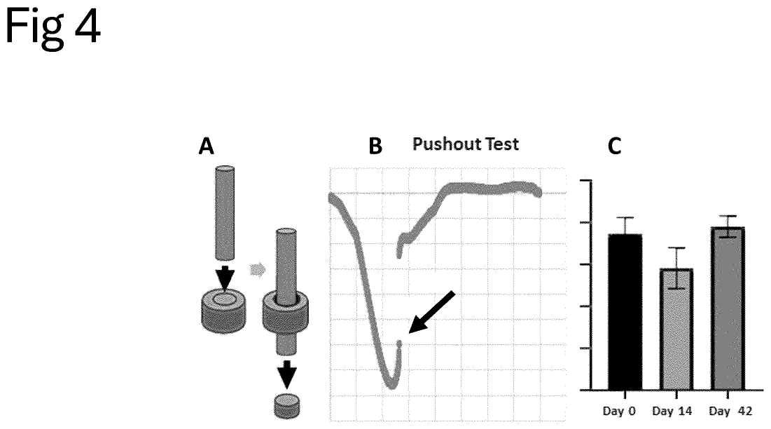

FIG. 5 shows SafraninO-stained histological sections of human osteochondral tissue taken from donor allografts subjected to full thickness cartilage injuries using a dermal punch as before, and then (A) pre-treated for 2 days with collagenase+hyaluronidase followed by 28 days maintenance in control media or (B) pre-treated for 2 days in control media (Mock) followed by 28 days of intermittent HBEGF treatment. Views are sagittal; the injuries are boxed. In (A) note the very prominent surface/superficial zone representing loss of SafraninO staining (ie, proteoglycan loss) caused by the enzyme pre-treatment (light gray area in upper third of explant, demarcated by vertical white line). This contrasts to the control explant (B) which received mock pretreatment followed by intermittent HB-EGF treatment for 28 days, in which SafraninO staining is robust throughout the cartilage layers. Note also the lack of injury healing following enzyme pre-treatment in (A) compared to partial seamless healing of the injury in (B) (white arrow).

FIG. 6 shows the standard tissue bank protocol for human osteochondral allograft storage and testing. Following harvest from a donor, the tissue bank carries out FDA-mandated serological and bacterial screening tests which typically require about 14 days to complete. After completion, the graft is size matched to a recipient patient, the recipient surgeon is notified to schedule surgery, and the graft is shipped.

FIG. 7 shows SafraninO-stained histological sections of explants of goat osteochondral tissue taken from (A,B) load-bearing and (C, D) non-load bearing regions of the femoral condyle and maintained for 28 days cither in (A, C) control media or (B, D) in media supplemented with HBEGF in an accelerated intermittent regimen in which five cycles of HB-EGF were administered (alternating with control media) over a 14 day period. As shown in (B), articular cartilage in an explant of load-bearing region maintained in the accelerated intermittent HBEGF regimen displays anabolic responses including markedly robust Safranin-O staining (gray shading in the cartilage midzone region indicated by an asterisk in B) and increased cell density (white arrows in B) compared to load-bearing cartilage maintained in control media (asterisk and arrow in A). In contrast, as shown in (D), articular cartilage in an explant of non-load bearing region did not display anabolic responses to the 14-day HB-EGF regimen as indicated by comparable SafraninO staining (compare area around the asterisks in D vs C) and no overt increased in cellularity.

FIG. 8 shows sections of the articular cartilage of osteochondral explants immunostained to detect presence of endogenous HB-EGF. The explants were either not injured (A) or were subjected to a superficial scalpel scratch injury (B-E) and then maintained in vitro in control media-no treatment-for 30 minutes to 6 days before harvesting (B=0-30 min, C=2 days, D=4 days; E=6 days). The injury model is diagrammed in (F) showing that the scalpel scratch is only as deep as the superficial zone and is not a full thickness injury. Compared to non-injured explants at the start of culture (A), superficial zone wounding initiates a rapid prolonged increase in local HBEGF produced by superficial zone cells occurring in 30 minutes of injury (compare B to A) and which is progressively increased at 2, 4 and 6 days later (compare C to B, D to C and E to D). (G, H) are graphs showing (G) the number of HB-EGF positive cells and (H) the extent of HB-EGF positive matrix in the superficial zone relative to the height of the articular cartilage, which are significantly increased in injured explants over time.

FIG. 9 shows sections of the articular cartilage of osteochondral explants immunostained to detect presence of lubricin, a superficial zone marker. The explants were either not injured (A) or subjected to superficial scalpel scratch injury (B-D) as before; and were either maintained in control media after injury for (B) 7 days, (C) 14 days or (E) 21 days; or in HBEGF continuously for 21 days (D). In the uninjured explant (A), note that lubricin staining is present throughout the matrix surrounding the superficial zone cells (medium gray area demarcated by vertical line). However, after injury and subsequent maintenance of the explants in control media for (B) 7 days, (C) 14 days and (E) 21 days, note that lubricin is progressively lost from the superficial zone matrix and that a region of acellular matrix appears adjacent to the injury where chondrocytes have died (asterisk). However, in the explant maintained in HBEGF for 21 days post-injury, lubricin staining is intense along the upper margins of the cut, and proliferating chondrocytes (doublets) are observed in the adjacent matrix (arrows in D); and these anabolic signs are accompanied by narrowing of the injury gap.

FIG. 10 shows the surgical approach and histological outcome of scaffold-mediated cartilage repair in a rabbit model in vivo. Panel (a) shows the full thickness osteochondral defect created surgically in the rabbit femoral condyle (white arrow); (b) shows the scaffold before implantation (inset in b) and the scaffold implanted into the defect site (black arrow). (c-f) are sections of the defect site 6 weeks later, stained with SafraninO to detect new cartilage matrix (c,e) or von Kossa (d,f) to detect bone. The shape of the original scaffold is outlined. In (c,d), note that no cartilage formation has occurred following implantation of the scaffold alone. In (c,f) note the appearance of SafrninO-stained cartilage matrix in the upper portion of the scaffold (arrows in c) that nearly bridge the gap, and new bone formation at the bottom of the scaffold (arrows in f).

FIG. 11 shows articular cartilage superficial zone (SZ) chondrocyte cell chemotaxis in response to HB-EGF. (A) is a diagram of the chemotaxis assay system. Chondrocytes (isolated from bovine articular cartilage) are contained within the matrix-filled central well but can travel in three-dimensions in response to the chemotaxis gradient contained in one of the adjacent chambers (shown as gray shading). The direction of potential cell movement along x,y axes is shown by arrows (gray=parallel to the gradient or towards it; black=perpendicular to the gradient or away from it). (B-H) Trajectories taken by SZ cells plotted on x,y coordinates over a 24 hour period in the absence of a gradient (B) or in response to gradients of 5, 10, or 50 ng/ml HB-EGF (B,-D) or SDF-1a (F-H). Trajectories moving towards the gradients are shown in gray; those moving away are shown in black; the relative proportions of trajectories moving in each direction are shown by the (%). In the absence of a gradient (E) there is no preferential directed migration. In the presence of gradients of HB-EGF (B-D) or SDF1a (F-H), there are 2-4 times more trajectories moving towards the gradients; this difference was significant (p<0.05) in response to gradients of 5 ng/ml HB-EGF (B); to reach a significant difference in response to SDF-1a, a gradient 10 fold greater was required (50 ng/ml SDF-1α (H). The average movement of all cells in each group is represented by the Center of Mass (white dot). Note the Center of Mass is displaced towards the growth factor gradient only in the 5 ng/ml HB-EGF group (C), representing coordinated movement.

FIG. 12 shows velocity (A) and FMI (Forward Migration Index) (B) of superficial zone chondrocyte chemotaxis. In (A), velocity is calculated as the total trajectory length traveled in 24 hours. For each group, average SZ cell velocity±SD is shown in response to gradients of 5, 10, or 50 ng/ml HB-EGF (black bars) or SDF-1a (gray bars); or no growth factor (negative control, open bar). Velocity is slowest in response to a gradient of 5 ng/ml HB-EGF; velocity is faster in response to a gradient of 5 ng/ml SDF1a but this difference is not significant (ns). Velocities in response to HB-EGF gradients at 10 and 50 ng/ml are significantly slower than in response to SDF-1a gradients at the same concentrations (**p<0.01; ***p<0.001). Velocity in the absence of any gradient (Negative Control) reflects random movement (chemokinesis). In (B) FMI describes efficiency of cell movement; FMI-y is the distance traveled parallel (towards) the gradient relative to the total distance. FMI-y values are shown for SZ cell movement over a 24-hour period in the presence of a gradient of 5, 10, or 50 ng/ml HB-EGF (black bars) or SDF-1a (gray bars); or no growth factor (negative control, open bar). FMI-y is greatest in response to the lowest concentration of HB-EGF (5 ng/ml); FMI-y values are significantly lower in all other groups except the highest concentration of SDF1a (50 ng/ml, ns). (*p<0.05)

FIG. 13 shows Histogram plots depicting the relative frequencies of trajectories taken by SZ cells over a 24-hour period in the absence of a gradient (A) or presence of gradients of 5, 10, or 50 ng/ml HB-EGF (B-D). Trajectories were converted to migration angles by measuring the angle of the straightest path from start (0) to end point relative to x,y axes, and migration angles were grouped into 10 degree intervals with the area of each wedge indicating the number of cells moving along a particular trajectory. In the absence of growth factor (A, Negative Control), most migration angles are clustered in quadrants representing opposite or perpendicular movement. In the presence of 5, 10, or 50 ng/ml HB-EGF (B-D) most migration angles are clustered among directional quadrants representing forward or parallel movement in relation to the concentration gradient. Note that only in the presence of 5 ng/ml HBEGF (B) are there no migration angles opposite to the concentration gradient (*).

FIG. 14 shows whole mount views of murine mesenchymal micromass cultures stained with Alcian blue, a cartilage marker and examined at day 7. (A) shows the spontaneous formation of chondrocyte nodules that occurs in this system after 7 days. (B) shows that when HBEGF is provided to the mesenchymal cells at the start of culture, chondrogenic differentiation is nearly completely impaired when examined at day 7 (cells are present, but they do not stain with Alcian blue). (C) shows that surprisingly, when provided after the cells had already entered the cartilage lineage, which occurs around day 4, chondrogenic differentiation was markedly promoted by HBEGF, as measured by size of the micromass diameter, abundance of cartilage nodules and its nearly uniform intensity of Alcian blue staining, a readout of new cartilage matrix synthesis, at day 7.

### DETAILED DESCRIPTION

### I. Methods for Restoring, Repairing, or Regenerating Cartilage in a Subject or Patient in Need Thereof

In one embodiment, the present disclosure relates to a method for restoring, repairing, or regenerating cartilage in a subject or patient in need thereof. In some embodiments, the method comprises restoring, repairing, or regenerating cartilage in at least one joint (e.g., a knee, a hip, a shoulder, an elbow, a wrist, an ankle, a digit, a spine, a temporomandibular joint, or any combination thereof). In some embodiments, the subject or patient is a mammal. In some embodiments, the subject or patient is suffering from a traumatic focal chondral or osteochondral injury. In some embodiments, the subject or patient is suffering from a degenerative disease or an inflammatory disease of at least one joint (e.g., a knee, a hip, a shoulder, an elbow, a wrist, an ankle, a digit, a spine, a temporomandibular joint, or any combination thereof). In yet other embodiments, the subject or patient is suffering from osteoarthritis. In still further embodiments, the subject or patient is a human. In still other embodiments, the subject or patient is suffering from osteochondritis dissecans. In some embodiments, the present disclosure relates to a method for restoring cartilage (e.g., such as in a joint) in a subject or patient in need thereof. In other embodiments, the present disclosure relates to a method for repairing cartilage (e.g., such as in a joint) in a subject or patient in need thereof. In still other embodiments, the present disclosure relates to a method for regenerating cartilage in a subject or patient in need thereof. As used herein, the term “restoring” or “restore” when used in connection with cartilage, refers to a procedure or process that simulates the growth or formation of new cartilage, such as in a joint (e.g., a knee, a hip, a shoulder, an elbow, a wrist, an ankle, a digit, a spine, a temporomandibular joint, or any combination thereof). In still other embodiments, the present disclosure relates to a method for maintaining the health or status of existing cartilage in a joint (e.g., a knee, a hip, a shoulder, an elbow, a wrist, an ankle, a digit, a spine, a temporomandibular joint, or any combination thereof) of a subject or patient in need thereof. As used herein, the term “repairing” or “repair” when used in connection with cartilage, refers to a procedure or process that helps to restore damaged cartilage in or around a joint (e.g., a knee, a hip, a shoulder, an elbow, a wrist, an ankle, a digit, a spine, a temporomandibular joint, or any combination thereof). As used herein, the term “regenerating” or “regeneration” as used in connection with cartilage, refers to a procedure or process that stimulates new cartilage growth or deposits (e.g., such as by injection or implantation) in a damage area, such as a joint (e.g., a knee, a hip, a shoulder, an elbow, a wrist, an ankle, a digit, a spine, a temporomandibular joint, or any combination thereof).

In some embodiments, the method involves providing a composition for injection or implantation in one or more joints (e.g., a knee, a hip, a shoulder, an elbow, a wrist, an ankle, a digit, a spine, a temporomandibular joint, or any combination thereof) of a subject or a patient, such as, for example, a mammal. In some embodiments, the subject or mammal is suffering from a traumatic focal chondral or osteochondral injury. In other embodiments, the subject or patient is suffering from osteoarthritis. In still further embodiments, the subject or patient is a human. In still other embodiments, the subject or patient is suffering from osteochondritis dissecans. In some embodiments, the composition comprises a tissue, a natural or synthetic scaffold or combination thereof, a hydrogel, a membrane, a particle (e.g., such as a sphere, microsphere, microparticle, nanoparticle or any combination thereof), chondrocytes or chondrogenic cells, or any combination of a tissue, natural or synthetic scaffold or combination thereof, hydrogel, membrane, particle, and/or chondrocytes or chondrogenic cells. In some embodiments, the composition comprises at least one of a tissue, a natural or synthetic scaffold or a combination thereof, a hydrogel, a membrane, a particle, chondrocytes or chondrogenic cells, or any combination thereof. In some embodiments, the composition comprises at least two of a tissue, a natural or synthetic scaffold or a combination thereof, a hydrogel, a membrane, a particle, chondrocytes or chondrogenic cells, or any combination thereof. In some embodiments, the composition comprises at least three of a tissue, a natural or synthetic scaffold or a combination thereof, a hydrogel, a membrane, a particle, chondrocytes or chondrogenic cells, or any combination thereof. In some embodiments, the composition comprises at least four of a tissue, a natural or synthetic scaffold or a combination thereof, a hydrogel, a membrane, a particle, chondrocytes or chondrogenic cells, or any combination thereof. In some embodiments, the composition comprises at least five of a tissue, a natural or synthetic scaffold or a combination thereof, a hydrogel, a membrane, a particle, chondrocytes or chondrogenic cells, or any combination thereof. In some embodiments, the composition comprises at least six of a tissue, a natural or synthetic scaffold or a combination thereof, a hydrogel, a membrane, a particle, chondrocytes or chondrogenic cells or any combination thereof. In other embodiments, the composition comprises one or more sutures (dissolvable and/or non-dissolvable), one or more stables, or any combination thereof.

In some embodiments, the composition comprises a tissue, a natural or synthetic scaffold or combination thereof, a hydrogel, a membrane, chondrocytes or chondrogenic cells, or any combination of a tissue, natural or synthetic scaffold or combination thereof, hydrogel, membrane, and/or chondrocytes or chondrogenic cells, but does not contain a particle. In some embodiments, the composition comprises a tissue, a natural or synthetic scaffold or combination thereof, a hydrogel, a membrane, chondrocytes or chondrogenic cells, or any combination of a tissue, natural or synthetic scaffold or combination thereof, hydrogel, membrane, and/or chondrocytes or chondrogenic cells but does not contain a sphere. In some embodiments, the composition comprises a tissue, a natural or synthetic scaffold or combination thereof, a hydrogel, a membrane, chondrocytes or chondrogenic cells, or any combination of a tissue, natural or synthetic scaffold or combination thereof, hydrogel, membrane and/or chondrocytes or chondrogenic cells, but does not contain a microsphere. In some embodiments, the composition comprises a tissue, a natural or synthetic scaffold or combination thereof, a hydrogel, a membrane, chondrocytes or chondrogenic cells, or any combination of a tissue, natural or synthetic scaffold or combination thereof, hydrogel, membrane, and/or chondrocytes or chondrogenic cells, but does not contain a microparticle. In some embodiments, the composition comprises a tissue, a natural or synthetic scaffold or combination thereof, a hydrogel, a membrane, chondrocytes or chondrogenic cells, or any combination of a tissue, natural or synthetic scaffold or combination thereof, hydrogel, membrane, and/or chondrocytes or chondrogenic cells, but does not contain a nanoparticle.

The tissue, natural or synthetic scaffold, or combination thereof, hydrogel, membrane, particle (e.g., sphere, microsphere, microparticle, nanoparticle, or any combination thereof), chondrocytes or chondrogenic cells, or any combination thereof is treated (e.g., pre-treated), coated, impregnated and/or embedded with at least one natural growth factor using techniques known in the art. For example, in some embodiments, the tissue, natural or synthetic scaffold, or combination thereof, hydrogel, membrane, particle, chondrocytes or chondrogenic cells, or any combination thereof is soaked with the at least one natural growth factor or soaked in the presence of the at least one natural growth factor (e.g., when the at least one natural growth factor is provided as a liquid). In other embodiments, the at least one natural growth factor is sprinkled, such as in the form of a powder or nanoparticles, on to the tissue, natural or synthetic scaffold, or combination thereof, hydrogel, membrane, particle, chondrocytes or chondrogenic cells, or any combination thereof. In still other embodiments, when the composition contains a scaffold, hydrogel, membrane, or particle, the scaffold, hydrogel, membrane, or particle can be manufactured to include the at least one growth factor as part of the structure of the scaffold, hydrogel, membrane, or particle. Depending on the nature of the composition, the at least one growth factor is absorbed or taken up by the composition (e.g., in the case of tissue and/or cells), coated on the surface of the composition (e.g., a natural or synthetic scaffold or combination thereof, a hydrogel, membrane, particle, or any combination thereof), and/or is included as part of the structure of the composition (e.g., a scaffold, hydrogel, membrane, or particle).

Any suitable natural growth factors can be used, some non-limiting examples are, heparin-binding epidermal growth factor (HB-EGF), a BMP (bone morphogenic protein), epidermal growth factor (EGF), transforming growth factor alpha (TGF-α), amphiregulin (AREG), betacellulin (BTC), epiregulin (EREG), epigen, neuregulin 1-4 (NRG1-4) and isoforms thereof, or any combinations thereof.

In some embodiments, the at least one natural growth factor is HB-EGF.

In still other embodiments, the at least one natural growth factor is a BMP. In still other embodiments, the BMP is BMP2, BMP3, BMP3B, BMP4, BMP5, BMP6, BMP7, BMP8, BMP8B, BMP9, BMP10, BMP12, BMP13, BMP14, BMP 15, or any combination thereof. In some embodiments, the BMP is BMP2. In yet other embodiments, the BMP is BMP3. In some embodiments, the BMP is BMP9. In yet further embodiments. the BMP is at least two of BMP2, BMP3, and BMP9.

In still other embodiments the at least one natural growth factor used in the composition is HB-EGF and the composition further comprises at least one growth factor which is BMP2. In still other embodiments the at least one natural growth factor used in the composition is HB-EGF and the composition further comprises at least one growth factor which is BMP3. In still other embodiments, the at least one natural growth factor used in the composition is HB-EGF and the composition further comprises at least one growth factor which is BMP9. In still yet other embodiments, the at least one natural growth factor used in the composition is HB-EGF and the composition further comprises at least two of BMP2, BMP3 and BMP9.

It was discovered that the use of at least one natural growth factor (such as HB-EGF and/or at least one BMP) in combination with a tissue, natural or synthetic scaffold, or combination thereof, a hydrogel, a membrane, particle (e.g., sphere, microsphere, microparticle, nanoparticle, or any combination thereof), chondrocytes or chondrogenic cells, or any combination thereof, improves repair by enhancing the ability of the tissue, natural or synthetic scaffold or combination thereof, hydrogel, membrane, particle, chondrocytes or chondrogenic cells, or any combination thereof, to recruit cells to the injection or implant site and/or by promoting integrative healing and formation of durable replacement cartilage tissue. It was further discovered that the use of at least one natural growth factor can improve cell-based repair (MACI) by making the expansion step more efficient and/or by promoting formation of durable cartilage tissue at an injection or implant site.

In some embodiments, the at least one natural growth factor can be added to composition containing a tissue, natural or synthetic scaffold, or combination thereof, hydrogel, membrane, particle, chondrocytes or chondrogenic cells, or any combination thereof, as an additive. For example, if the composition contains a tissue, natural or synthetic scaffold or combination thereof, or membrane, particle, or chondrocytes or chondrogenic cells, that is stored on a media or in some type of receptacle (e.g., container, bottle, vial, flask, or any combination thereof), the at least one natural growth factor can be added directly to the media or receptacle. In some embodiments, the at least one natural growth factor is supplied as a liquid and is poured into or onto the media or receptacle containing the tissue, natural or synthetic scaffold or combination thereof, hydrogel, membrane, particle, or chondrocytes or chondrogenic cells. In other embodiments, the at least one natural growth factor is supplied as a powder or particles, which are sprinkled or poured onto the media or receptacle containing the tissue, natural or synthetic scaffold or combination thereof, membrane, particle, or chondrocytes or chondrogenic cells. In still other embodiments, a tissue, natural or synthetic scaffold or combination thereof, or membrane, particle, or chondrocytes or chondrogenic cells, can be immersed or “dipped” into a liquid containing at least one natural growth factor (e.g., for a period fat at least 30 seconds, at least 1 minutes, at least 2 minutes, at least 3 minutes, at least 4 minutes, at least 5 minutes, at least 15 minutes, at least 30 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 36 hours, at least 48 hours, at least 3 days, at least 4 days, at least 5 days, at least 6 days, at least 7 days, at least 8 days, at least 9 days, at least 10 days, at least 11 days, at least 12 days, at least 13 days, at least 14 days, at least 3 week, at least 4 weeks, at least 5 weeks, at least 6 weeks, at least 7 weeks, at least 8 weeks, at least 9 weeks, or at least 10 weeks). In some embodiments, when supplied as a liquid, the amount of at least one natural growth factor that can be used is about 1 ng/ml to about 500 ng/mL. In other embodiments, when supplied as a liquid, the amount of at least one natural growth factor that can be used is about 1 ng/mL to about 400 ng/mL. In still other embodiments, when supplied as a liquid, the amount of at least one natural growth factor that can be used is about 1 ng/mL to about 300 ng/mL. In still other embodiments, when supplied as a liquid, the amount of at least one natural growth factor that can be used is about 1 ng/mL to about 200 ng/ml. In still other embodiments, when supplied as a liquid, the amount of at least one natural growth factor that can be used is about 1 ng/mL to about 100 ng/mL.

In some embodiments, the composition comprises a hydrogel. In some embodiments, the at least one natural growth factor is supplied as a liquid and is combined with the hydrogel either during preparation of the hydrogel or once the hydrogel has been prepared. In other embodiments, the at least one natural growth factor is supplied as a powder, which is sprinkled or poured in or onto the hydrogel either during the preparation of the hydrogel or once the hydrogel has been prepared. In some embodiments, when supplied as a liquid, the amount of at least one natural growth factor that can be used is about 1 ng/mL to about 500 ng/ml. In other embodiments, when supplied as a liquid, the amount of at least one natural growth factor that can be used is about 1 ng/mL to about 400 ng/mL. In still other embodiments, when supplied as a liquid, the amount of at least one natural growth factor that can be used is about 1 ng/ml to about 300 ng/mL. In still other embodiments, when supplied as a liquid, the amount of at least one natural growth factor that can be used is about 1 ng/mL to about 200 ng/mL. In still other embodiments, when supplied as a liquid, the amount of at least one natural growth factor that can be used is about 1 ng/ml to about 100 ng/ml.

In some embodiments, the tissue, natural or synthetic scaffold, or combination thereof, hydrogel, membrane, particle, chondrocytes or chondrogenic cells, or any combination thereof is treated (e.g., pre-treated), coated, and/or impregnated with heparin-binding epidermal growth factor (HB-EGF), epidermal growth factor (EGF), BMP (bone morphogenic protein, such as BMP2 and BMP9), transforming growth factor alpha (TGF-α), amphiregulin (AREG), betacellulin (BTC), epiregulin (EREG), epigen, neuregulin 1-4 (NRG1-4) and isoforms thereof, or any combinations thereof.

In yet other embodiments, the tissue, natural or synthetic scaffold, or combination thereof, hydrogel, membrane, particle, chondrocytes or chondrogenic cells, or any combination thereof is treated (e.g., pre-treated), coated, and/or impregnated with heparin-binding epidermal growth factor (HB-EGF). In other embodiments, the tissue, natural or synthetic scaffold, or combination thereof, hydrogel, membrane, particle, chondrocytes or chondrogenic cells, or any combination thereof is treated (e.g., pre-treated), coated, and/or impregnated with epidermal growth factor (EGF). In other embodiments, the tissue, natural or synthetic scaffold, or combination thereof, hydrogel, membrane, particle, chondrocytes or chondrogenic cells, or any combination thereof is treated (e.g., pre-treated), coated, and/or impregnated with) transforming growth factor alpha (TGF-α). In other embodiments, the tissue, natural or synthetic scaffold, or combination thereof, hydrogel, membrane, particle, chondrocytes or chondrogenic cells, or any combination thereof is treated (e.g., pre-treated), coated, and/or impregnated with amphiregulin (AREG). In other embodiments, the tissue, natural or synthetic scaffold, or combination thereof, hydrogel, membrane, particle, chondrocytes or chondrogenic cells, or any combination thereof is treated (e.g., pre-treated), coated, and/or impregnated with betacellulin (BTC). In other embodiments, the tissue, natural or synthetic scaffold, or combination thereof, hydrogel, membrane, particle, chondrocytes or chondrogenic cells, or any combination thereof is treated (e.g., pre-treated), coated, and/or impregnated with epiregulin (EREG). In other embodiments, the tissue, natural or synthetic scaffold, or combination thereof, hydrogel, membrane, particle, chondrocytes or chondrogenic cells, or any combination thereof is treated (e.g., pre-treated), coated, and/or impregnated with epigen. In other embodiments, the tissue, natural or synthetic scaffold, or combination thereof, hydrogel, membrane, particle, chondrocytes or chondrogenic cells, or any combination thereof is treated (e.g., pre-treated), coated, and/or impregnated with neuregulin 1-4 (NRG1-4) and isoforms thereof. In yet other embodiments, the tissue, natural or synthetic scaffold, or combination thereof, hydrogel, membrane, particle, chondrocytes or chondrogenic cells, or any combination thereof is treated (e.g., pre-treated), coated, and/or impregnated with bone morphogenetic protein (BMP) family member (such as BMP2 and BMP9). In yet other embodiments, the tissue, natural or synthetic scaffold, or combination thereof, hydrogel, membrane, particle, chondrocytes or chondrogenic cells, or any combination thereof is treated (e.g., pre-treated), coated, and/or impregnated with a BMP (e.g., such as BMP2 and BMP9) and heparin-binding epidermal growth factor (HB-EGF).

In some embodiment, the tissue can be from any suitable origin, for example, human, pig, or genetically modified pig. In some other embodiments, the tissue is a combination of bone and cartilage (e.g., osteochondral tissue), cartilage (e.g., chondral tissue), meniscus, or any combination thereof. When bone and cartilage (e.g., osteochondral tissue) or only cartilage (e.g., chondral tissue) is used, this tissue can be used in any form or condition, such as, for example, living (e.g., alive), cryopreserved, frozen, decellularized, minced, powder, or any combinations thereof. In some embodiments, the bone and cartilage (e.g., osteochondral tissue), cartilage (e.g., chondral tissue) or meniscus is obtained from any source (e.g., a mammal). In some embodiments, the bone and cartilage (e.g., osteochondral tissue), cartilage (e.g., chondral tissue) or meniscus is obtained from pigs. In some other embodiments, the bone and cartilage (e.g., osteochondral tissue), cartilage (e.g., chondral tissue) or meniscus is obtained from genetically modified pigs (e.g., pigs that have been engineered to provide tissue for use in human engraftment, such, as for example, genetically modified pigs that have had one or more genes knocked out (e.g., meaning expression of one or more genes has been lost in its entirety or suppressed) or overexpressed) In still other embodiments, the bone and cartilage (e.g., osteochondral tissue), cartilage (e.g., chondral tissue) or meniscus is obtained from a human, either living or deceased. In still other embodiments, the bone and cartilage (e.g., osteochondral tissue), cartilage (e.g., chondral tissue) or meniscus is obtained from a human, living or deceased, with osteoarthritis or other joint degeneration.

In some embodiments, natural scaffolds can be made from materials such as collagen, aragonite, gelatin, elastin, hyaluronic acid (HA), chitosan (CH), chondroitin sulfate (CS), agarose, alginate, cellulose, fibrin or any combinations thereof. Synthetic scaffolds can be made from materials such as poly(ethylene glycol) (PEG), polycaprolactone (PCL), polylactic acid (PLA), polyurethane (PU), poly(glycolic acid) (PGA), polyethersulfone (PES), poly-p-dioxanone (PDS), poly(l-lactide) (PLLA), polylactic-co-glycolic acid (PLGA), polycaprolactone-polyethylene glycol (PCEC), polysulfone or any combinations thereof. Hybrid scaffolds comprise a mixture of materials used to make natural and synthetic scaffolds. Examples of natural, synthetic and hybrid scaffolds that can be used are provided in the below table:

| Scaffold Name | Component | Type of Scaffold |
|---|---|---|
| BioSeed ®-C (Biotissue) | PGA/PLA, PDS | Synthetic |
| Spongy PU scaffold | PU | Synthetic |
| NSP-PCL scaffold | PCL | Synthetic |
| RO45 3DHC | PCL | Synthetic |
| Polysulphonic scaffold | PES | Synthetic |
| PLLA-100 scaffolds | PLLA | Synthetic |
| PLCL-2 scaffold | PLCL | Synthetic |
| Bioglass | Comprises silicon dioxide, | Natural |
| | sodium oxide, calcium oxide, | |
| | and phosphorous pentoxide | |
| Chondrotissue ® | PGA, HA | Hybrid |
| IC scaffold | PLGA, COL | Hybrid |
| Gel/PCEC-TGFβ1 hydrogel | Gelatin, PCEC, TGFβ1 | Hybrid |
| scaffold | | |
| PLCL-COLI | PLCL, COL | Hybrid |
| C2C1H scaffold | PLA, COL, CH | Hybrid |
| ECM-PLGA scaffold | PLGA, ECM | Hybrid |
| PCL/COL1 | PCL, COL | Hybrid |
| CH/PLLA/PC scaffold | PLLA, CH, PC | Hybrid |
| Chitosan-modified PLCL | PLCL, CH | Hybrid |
| scaffold | | |
| CSMA/PECA/GO (S2) | CSMA, MPEG-PCL-AC | Hybrid |
| scaffold | (PECA), GO | |
| silk | silk | Natural |
| Hyaluronan | | Natural |
| Hydroxyapatite | | Natural |
| Chitosan | | Natural |
| Argonite with hyaluronan | Calcium carbonate in | Natural composition, made |
| Agili-C, Cartiheal | the aragonite crystalline form | synthetically |
| | as a bone phase, combined | |
| | with a superficial cartilage | |
| | phase composed of modified | |
| | aragonite and hyaluronic acid | |
| Hyalofast ®(Anika) | Benzyl ester of hyaluronic | Natural |
| | acid | |
| NeoCart ®(Histogenics) | Bovine type I collagen | Natural |
| ChondroGide(Geistlich) | Type I/III collagen | Natural |
| ACI-MaixTM (MACI) | Type I/III collagen | Natural |
| Cartipatch ®(Xizia Biotech) | Agarose and alginate | Natural |
| NOVOCART ® 3D- | Type I collagen, chondroitin | Natural |
| AesculapOrthopaedics | sulfate | |
| (BBraun | | |
| CaReS ®(Arthrokinetics) | Type I collagen gel | Natural |
| CARTISTEM ® (Medipost) | Hyaluronic acid | Natural |

CH—chitosan; COL—collagen, PU—polyurethane; PC—pectin based; PDS—poly-p-dioxanone; CS—chondroitin sulfate; CSMA—methacrylated chondroitin sulfate; HA—hyaluronic acid; PEG—poly(ethylene glycol); PCL—polycaprolactone; PLA—polylactic acid; PLLA—poly(l-lactide); PGA—poly(glycolic acid); PES—polyethersulfone; PLGA—polylactic-co-glycolic acid; PCEC—polycaprolactone-polyethylene glycol; ECM—extracellular matrix; PLCL—poly(l-lactide-co-ε-caprolactone); SCPL—solvent casting and particulate leaching method; AC—acryloyl chloride; GO—graphene oxide; PECA—poly(ethylene glycol) methyl ether-ε-caprolactone-acryloyl chloride.

In some embodiments, the membrane can be a collagen membrane (such as those obtained from pigs, bovine, humans, primates or other mammals), gelatin, elastin, poly(vinylalcohol)-based membranes, polyester (L-lactide-co-ε-caprolactone-PLCA) membranes, polyurethane membranes or any combinations thereof.

In some embodiments, the hydrogel can be made from one or more polymers. The one or more polymers can be natural polymers or synthetic polymers or a combination thereof. The natural polymers that can be used include collagen, gelatin, elastin, hyaluronic acid, chitosan, chondroitin sulfate, agarose, alginate, cellulose, fibrin, proteonucleotide-based polymers (e.g., DNA) or any combinations thereof. Synthetic polymers that can be used be made from materials such as poly(ethylene glycol) (PEG), poly(vinyl alcohol) (PVA), polylactic acid (PLA), polydioxanone (PDS), and poly(2-hydroxypropyl methacrylamide) (PHPMA), as well as copolymers such as poly(N-isopropyl acrylamide) (PNIPAAm), P(PEG-co-peptides), P(PLGA-co-serine) or any combinations thereof.

In some embodiments, the particle (e.g., sphere, microsphere, microparticle, nanoparticle, or any combination thereof) can be made from one or more natural materials such as collagen, silk, gelatin, elastin, hyaluronic acid (HA), chitosan (CH), chondroitin sulfate (CS), agarose, alginate, cellulose, fibrin, arogonite or any combinations thereof. In other embodiments, the particles can be from degradable and/or biodegradable polymers, such as poly(ethylene glycol) (PEG), polycaprolactone (PCL), polylactic acid (PLA), polyurethane (PU), polyether urethane, poly(glycolic acid) (PGA), polyethersulfone (PES), poly-p-dioxanone (PDS), poly(l-lactide) (PLLA), polylactic-co-glycolic acid (PLGA), ethyl cellulose, polycaprolactone-polyethylene glycol (PCEC), polysulfone or any combinations thereof. An example of a particle (e.g., sphere, microsphere, microparticle, nanoparticle, or any combination thereof) made from a combination of degradable and/or biodegradable polymers includes particles made from a combination of PLGA and PLLA.

In some embodiments, the composition can further comprise at least one heparinase, heparan sulfate, or a combination thereof to increase the bioavailability of the at least one natural growth factor to bind to its respective receptor. The heparinase and/or heparan sulfate can be added to the composition before or after the addition of the at least one natural growth factor. In some aspects, the heparinase and/or heparan sulfate can be added to the composition prior to the addition of the at least one natural growth factor to pre-treat the composition for the at least one natural growth factor. In some embodiments, the composition comprises HB-EGF and heparinase or heparan sulfate. In other embodiments, the composition comprises a BMP family member (e.g., BMP2 and/or BMP9) and heparinase and/or heparan sulfate. In still other embodiments, the composition comprises HB-EGF, at least one BMP family member (e.g., BMP2 and/or BMP9) and heparinase and/or heparan sulfate.

Once the tissue, natural or synthetic scaffold, or combination thereof, hydrogel, membrane, particle, chondrocytes or chondrogenic cells, or any combination thereof is treated (e.g., pre-treated), coated, impregnated and/or embedded with at least one natural growth factor, the composition (hereinafter “growth factor pre-treated composition”) can be injected and/or implanted into a joint of subject or patient in need of treatment thereof using techniques known in the art. In some embodiments, (MACI) Matrix Assisted Chondrocyte Implantation is used. In some embodiments, the growth factor pre-treated composition is injected or implanted to restore cartilage in the subject or patient. In other embodiments, the growth factor pre-treated composition is injected or implanted in the subject or patient to repair cartilage in the subject or patient. In still other embodiments, the growth factor pre-treated composition is injected or implanted to regenerate cartilage in the subject or patient. Specifically, it was discovered that when the growth factor pre-treated compositions described herein are injected or implanted in a subject or patient in need of restoring, repairing, or regenerating cartilage, that the composition promotes growth or seamless healing of articular cartilage in the subject or patient.

In still other embodiments, the methods disclosed herein provide improved compositions (e.g., therapeutics) and methods (e.g., treatments) for use in a variety of cartilage restoration procedures, such as surgical cartilage restoration procedures, microfracture, matrix-induced autologous chondrocyte implantation (MACI), debridement, meniscus repair, osteoarthritis treatment, osteochondral or chondral auto or allo grafting, articular fracture healing, growth plate fracture or injury healing in a subject, or patient, such as a pediatric patient.

### 2. Methods for Increasing the Shelf-Life of a Composition

In another embodiment, the present disclosure relates to method of increasing the shelf-life of a composition comprising an allograft, which can comprise only cartilage (e.g., a chondral allograft), a meniscus, or bone and cartilage (e.g., an osteochondral allograft) or an autograft, which comprise only cartilage (e.g., a chondral autograft), meniscus, or bone and cartilage (e.g., an osteochondral autograft), or any combination thereof. In some embodiments, the method comprises adding at least one natural growth factor to a composition comprising an osteochondral, meniscus, or chondral allograft, osteochondral, meniscus, or chondral autograft, or any combination thereof. In some embodiments, the at least one natural growth factor is heparin-binding epidermal growth factor (HB-EGF), epidermal growth factor (EGF), a bone morphogenetic protein (BMP) (such as BMP2 and BMP9), transforming growth factor alpha (TGF-α), amphiregulin (AREG), betacellulin (BTC), epiregulin (EREG), epigen, neuregulin 1-4 (NRG1-4) and isoforms thereof, or any combinations thereof. In some embodiments, the at least one natural growth factor is HB-EGF.

In some embodiments, the at least one natural growth factor can be added to composition containing osteochondral, meniscus, or chondral allograft or osteochondral, meniscus, or chondral autograft, or any combination thereof, as an additive. For example, if the composition containing the osteochondral, meniscus, or chondral allograft or osteochondral, meniscus, or chondral autograft, or any combination thereof is stored on a media or in some type of receptacle (e.g., container, bottle, bag, vial, flask, or any combination thereof), the at least one natural growth factor can be added directly to the media or receptacle. In some embodiments, the at least one natural growth factor is supplied as a liquid and is poured into or onto the media or receptacle containing the osteochondral or chondral allograft or osteochondral or chondral autograft, or any combination thereof. In other embodiments, the at least one natural growth factor is supplied as a powder, which is sprinkled or poured onto the media or receptacle. In some embodiments, when supplied as a liquid, the amount of at least one natural growth factor that can be used is about 1 ng/ml to about 10 mg/mL. In some embodiments, when supplied as a liquid, the amount of at least one natural growth factor that can be used is about 5 ng/ml to about 10 mg/mL. In some embodiments, when supplied as a liquid, the amount of at least one natural growth factor that can be used is about 25 ng/ml to about 10 mg/mL. In other embodiments, when supplied as a liquid, the amount of at least one natural growth factor that can be used is about 1 mg/mL to about 10 mg/mL. In still other embodiments, when supplied as a liquid, the amount of at least one natural growth factor that can be used is about 1 ng/mL to about 500 ng/mL. In still other embodiments, when supplied as a liquid, the amount of at least one natural growth factor that can be used is about 1 ng/mL to about 400 ng/ml. In still other embodiments, when supplied as a liquid, the amount of at least one natural growth factor that can be used is about 1 ng/ml to about 300 ng/mL. In still other embodiments, when supplied as a liquid, the amount of at least one natural growth factor that can be used is about 1 ng/ml to about 200 ng/mL. In still other embodiments, when supplied as a liquid, the amount of at least one natural growth factor that can be used is about 1 ng/mL to about 100 ng/mL.

In some embodiments, particles (spheres, microspheres, microparticles, nanoparticles, or any combinations thereof), treated (e.g., pre-treated), coated, impregnated and/or embedded with at least one natural growth factor (e.g., such as heparin-binding epidermal growth factor (HB-EGF)), as described in Section 1 herein, can be added to or included in the composition containing osteochondral or chondral allograft or osteochondral or chondral autograft, or any combination thereof to help protect the allograft or autograft from degradation. In some embodiments, spheres are added to or included in the composition containing osteochondral or chondral allograft or osteochondral or chondral autograft, or any combination thereof. In other embodiments, microspheres are added to or included in the composition containing osteochondral or chondral allograft or osteochondral or chondral autograft, or any combination thereof. In still other embodiments, microparticles are added to or included in the composition containing osteochondral or chondral allograft or osteochondral or chondral autograft, or any combination thereof. In still yet other embodiments, nanoparticles are added to or included in the composition containing osteochondral or chondral allograft or osteochondral or chondral autograft, or any combination thereof.

In some embodiments, when the at least one natural growth factor is added to the composition containing the osteochondral or chondral allograft or osteochondral or chondral autograft, or any combination thereof, the shelf-life of the composition (e.g., clinical shelf life) is increased for a period of at least one week to about eight weeks when compared to a composition comprising an osteochondral or chondral allograft, osteochondral or chondral autograft or combination thereof that does not contain at least one natural growth factor. For avoidance of any doubt, the at least one natural growth factor is added as an exogenous agent to the composition and is not included or provided as part of any media composition. The increase of the shelf-life of the composition (e.g., clinical shelf life) reduces waste and increases graft availability (e.g., supply) for surgical procedures. In other embodiments, when the at least one natural growth factor is added to the composition containing the osteochondral or chondral allograft or osteochondral or chondral autograft or any combination thereof, the shelf-life of the composition (e.g., clinical shelf life) is increased for a period of at least one week when compared to a composition comprising an osteochondral or chondral allograft, osteochondral or chondral autograft or combination thereof that does not contain at least one natural growth factor. In still other embodiments, when the at least one natural growth factor is added to the composition containing the osteochondral or chondral allograft or osteochondral or chondral autograft or any combination thereof, the shelf-life of the composition (e.g., clinical shelf life) is increased for a period of at least two weeks when compared to a composition comprising an osteochondral or chondral allograft, osteochondral or chondral autograft or combination thereof that does not contain at least one natural growth factor. In still other embodiments, when the at least one natural growth factor is added to the composition containing the osteochondral or chondral allograft or osteochondral or chondral autograft or any combination thereof, the shelf-life of the composition (e.g., clinical shelf life) is increased for a period of at least three weeks when compared to a composition comprising an osteochondral or chondral allograft, osteochondral or chondral autograft or combination thereof that does not contain at least one natural growth factor. In still other embodiments, when the at least one natural growth factor is added to the composition containing the osteochondral or chondral allograft or osteochondral or chondral autograft or any combination thereof, the shelf-life of the composition (e.g., clinical shelf life) is increased for a period of at least four weeks when compared to a composition comprising an osteochondral or chondral allograft, osteochondral or chondral autograft or combination thereof that does not contain at least one natural growth factor. In still other embodiments, when the at least one natural growth factor is added to the composition containing the osteochondral or chondral allograft or osteochondral or chondral autograft or any combination thereof, the shelf-life of the composition (e.g., clinical shelf life) is increased for a period of at least five weeks when compared to a composition comprising an osteochondral or chondral allograft, osteochondral or chondral autograft or combination thereof that does not contain at least one natural growth factor. In still other embodiments, when the at least one natural growth factor is added to the composition containing the osteochondral or chondral allograft or osteochondral or chondral autograft or any combination thereof, the shelf-life of the composition (e.g., clinical shelf life) is increased for a period of at least six weeks when compared to a composition comprising an osteochondral or chondral allograft, osteochondral or chondral autograft or combination thereof that does not contain at least one natural growth factor. In still other embodiments, when the at least one natural growth factor is added to the composition containing the osteochondral or chondral allograft or osteochondral or chondral autograft or any combination thereof, the shelf-life of the composition (e.g., clinical shelf life) is increased for a period of at least seven weeks when compared to a composition comprising an osteochondral or chondral allograft, osteochondral or chondral autograft or combination thereof that does not contain at least one natural growth factor. In still other embodiments, when the at least one natural growth factor is added to the composition containing the osteochondral or chondral allograft or osteochondral or chondral autograft or any combination thereof, the shelf-life of the composition (e.g., clinical shelf life) is increased for a period of at least eight weeks when compared to a composition comprising an osteochondral or chondral allograft, ostcochondral or chondral autograft or combination thereof that does not contain at least one natural growth factor.

In some embodiments, the composition can further comprise at least one heparinase, heparan sulfate, or a combination thereof to increase the bioavailability of the at least one natural growth factor to bind to its respective receptor. The heparinase and/or heparan sulfate can be added to the composition before or after the addition of the at least one natural growth factor. In some aspects, the heparinase and/or heparan sulfate can be added to the composition prior to the addition of the at least one natural growth factor to pre-treat the composition for the at least one natural growth factor.

In some embodiments, when HB-EGF was added to media of human osteochondral allografts in vitro, it was found that when implanted into a subject in need thereof, the HB-EGF promoted anabolic responses by the cartilage of the graft (as assessed by cell proliferation and matrix synthesis). In addition, the HB-EGF was found to promote measurable cartilage growth of the grafts in vitro (as assessed by the height of the cartilage and noncalcified cartilage zones). Additionally, it was also discovered that the addition of HB-EGF to the composition promoted integrative cartilage healing by the allografts of cartilage incisional injuries in vitro including full thickness cartilage injury. Moreover, the addition of HB-EGF was found to maintain the health of the grafts (as assessed by cell density and matrix deposition) at least one week past their clinical surgical shelf life (about 28 days versus 21 days). Finally, it was also discovered that when HB-EGF is used as a coating on a synthetic scaffold, the coated synthetic scaffold promotes repair of a focal articular cartilage defect in a rabbit model better than uncoated scaffold and the HB-EGF is also a potent stimulus for migration and chondrogenic differentiation by cartilage progenitor cells in vitro.

The present disclosure is illustrated and further described in more detail with reference to the following non-limiting examples. Section headings as used in this section and the entire disclosure herein are merely for organizational purposes and are not intended to be limiting.

### Example 1—Human Articular Cartilage Explants Treated with HBEGF Display Unexpected Dramatic Anabolic Growth and Healing Responses In Vitro

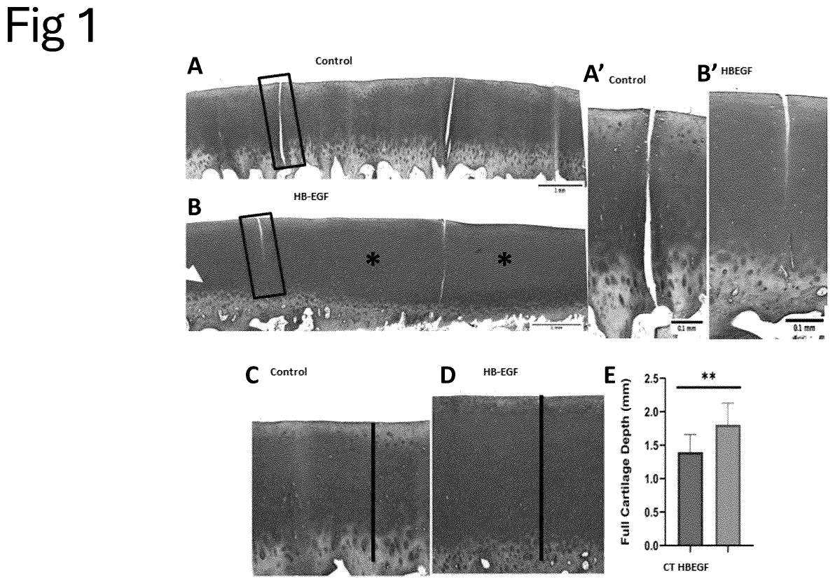

Described herein is a system that models OCA surgery, an approach for cartilage repair in patients with local cartilage damage. A challenge in this approach is that the grafts fail to heal into the patient's native cartilage tissue. Developing an approach to pre-treat or prime the grafts either during their in vitro storage prior to implantation, or immediately before surgery, to activate their latent regenerative potential, could fix this problem. In FIG. 1, explants of human osteochondral allografts were subjected to full thickness cartilage injury and then maintained for 28 days in vitro. The allografts were obtained about 7 days following harvest from a deceased but otherwise healthy male and female donors aged 14-38 years of age, making the final 28^{th} day of culture equivalent to day 35 post-mortem. Injured osteochondral allografts maintained in control media for 28 days remain similar to uncultured grafts (data not shown) and the injuries do not close. Grafts maintained in the presence of HBEGF added to the media (25-500 ng/ml) continuously are thicker, more cellular and safranin staining (an indicator of cartilage matrix deposition) is more robust indicating anabolic growth responses. Note that 28 days in the in vitro system (i.e., 35 days postmortem) is −14 days past typical storage discard which is 28 days postmortem, demonstrating utility of this treatment to extend osteochondral allograft shelf life. Remarkably, measurement of digital images showed that the thickness of the cartilage actually increased following HBEGF treatment in vitro. This is unprecedented for human adult articular cartilage in vitro. N=3 each group. Additionally, closure of the injury is truly seamless where it has healed. This is also unprecedented and never shown in an adult cow or human articular cartilage system before.

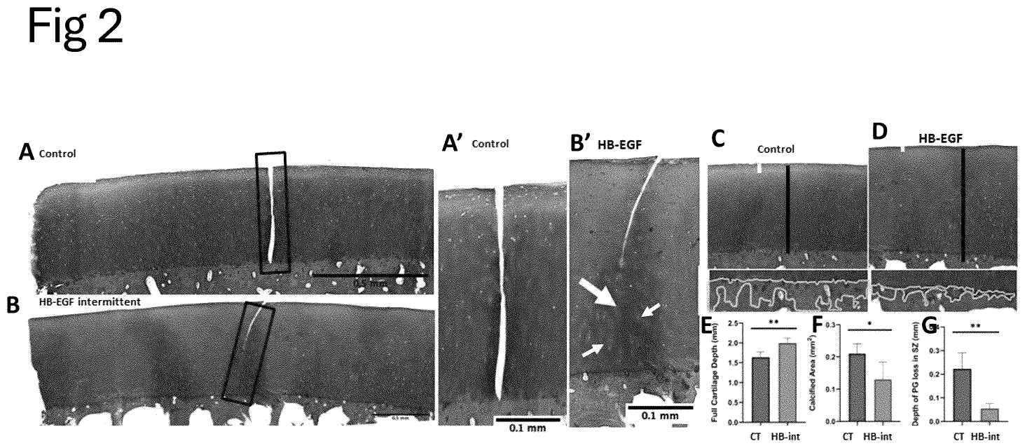

In FIG. 2, surprisingly, intermittent treatment with the HBEGF promoted even more robust anabolic growth and healing responses. Growth factor was added after 0-5 days control media, on a schedule of 5 days control, 3 days treated, repeat, for 28 days. Unexpectedly this regimen resulted in increased safranin positive matrix all the way to the top of the explants such that the immature matrix characteristic of the superficial zone was not present. This is an unprecedented anabolic response. Measurement of calcified and uncalcified portions of the cartilage revealed differential responses between. continuous and intermittent treatment, with calcified (hypertrophic) cartilage thickness being decreased following intermittent treatment. This suggests that hypertrophy is blocked which is also an anabolic response. The tissue is also more cellular/proliferative, further evidence of anabolic response to intermittent HBEGF. Note remarkable seamless healing of the cartilage at the arrow. N=3 each group.

### Example 2—Preventing HBEGF Interaction with HSPGs Enhances HBEGF Effects

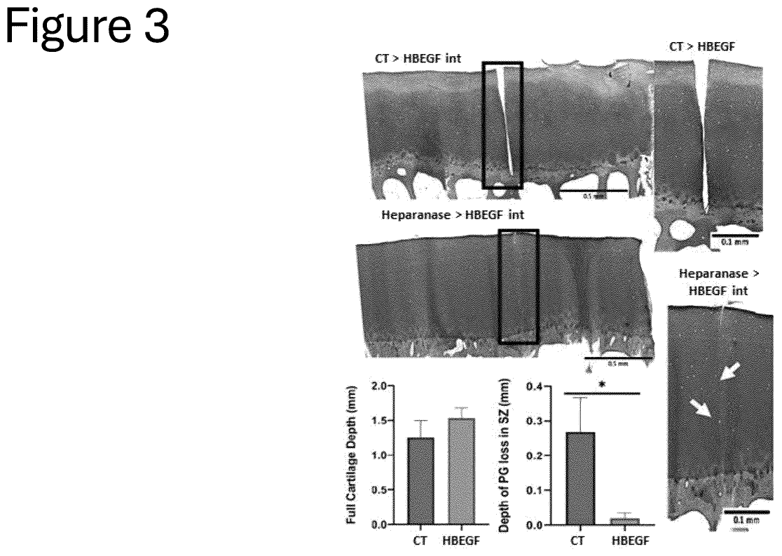

HB-EGF is a heparan binding protein which means it can bind to HSPG or heparan sulfated proteoglycans. Preventing this binding might increase HBEGF bioavailability for its receptors. In FIG. 3 explants were treated with heparanase or heparan sulfate (not shown) for 2 days to disrupt/prevent HBEGF-HSPG binding and then maintained the explants in either control or HBEGF for 28 days. N=3 each group. It was found that heparanase treated explants were much thicker and had healing of the injury all the way to the top of the explant, including through the superficial zone. Healing was remarkable and appeared to be truly seamless. The dramatic nature of this response was unexpected, as true seamless healing of injured human cartilage has to our knowledge never been previously reported. Additionally, fibrils were noted (not shown) in the explant.

Together, this shows that HBEGF promotes anabolic growth and integrative healing in human adult articular cartilage/ostcochondral tissue that suggests utility in improved engraftment by promoting in vivo healing and hence retention of OC allo/autografts in patients.

### Example 3—Human Articular Cartilage Healing Behavior Versus Bovine Articular Cartilage

Unexpectedly, human articular cartilage does NOT recapitulate the healing behavior of bovine articular cartilage. This is important because bovine joint cartilage has been considered the gold standard surrogate for human cartilage research because healthy human articular cartilage is difficult to obtain. As reported by Tam H K, Srivastava A, Colwell C W Jr, D'Lima D D., “In vitro model of full-thickness cartilage defect healing”, J Orthop Res., 2007 September; 25(9):1136-44, dermal punch injuries through the articular cartilage of bovine osteochondral explants undergo complete, spontaneous, healing in a matter of weeks in vitro in basal media (albeit with a scam still present). To determine if human cartilage can also heal spontaneously, mechanical strength testing by push out test was used to quantify functional cartilage integration in explants of injured bovine ostcochondral tissue maintained for various periods of time in control media. In controls—no treatment—there was no difference in force needed to push the core through from 0, 14, or 42 day control explants, this means no healing occurred in controls on a spontaneous basis even after an extended period in vitro. The data thus confirm important unexpected differences that are unappreciated/surprising between human and bovine articular cartilage. This discovery has important clinical considerations that emphasize how unusual it was to induce healing in human articular cartilage with HBEGF treatment.

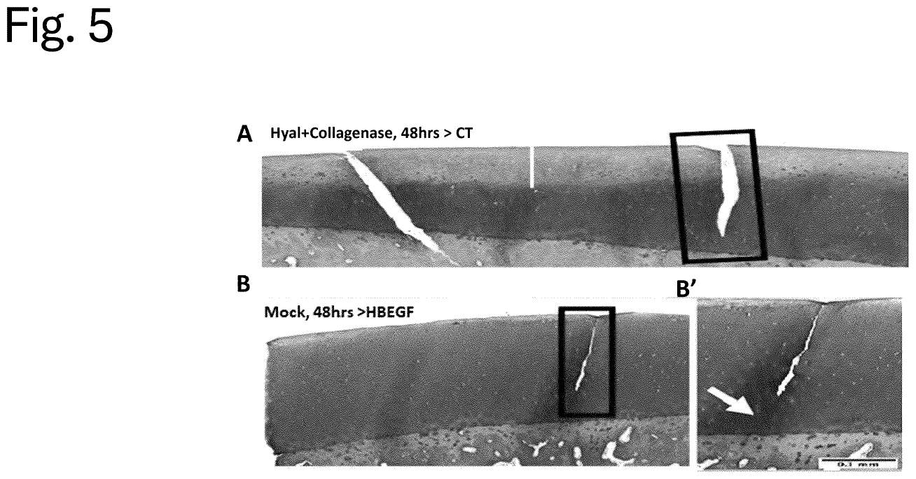

Further evidence supports distinct healing responses between humans and cows is provided in FIG. 5. van de Breevaart Bravenboer J, In der Maur C D, Bos P K, Feenstra L, Verhaar J A, Weinans H, van Osch G J., “Improved cartilage integration and interfacial strength after enzymatic treatment in a cartilage transplantation model”, Arthritis Res Ther., 2004; 6(5): reported that collagenase and hyaluronidase treatment of bovine osteochondral explants stimulate proliferation and appears to promote what the authors call integrative cartilage healing of an induced full thickness injury (although as shown in their figures it is noted that this healing is not seamless, nor complete). However, as shown in FIG. 5, similar collagenase and hyaluronidase treatment of human osteochondral explants did NOT improve healing responses of human articular cartilage after 28 days. This is unexpected and further suggests differences in healing responses between human cartilage and bovine cartilage, the current surrogate study system.

### Example 4—A Clinically Compliant, 14-Day HB-EGF Five-Cycle Pre-Treatment Regimen also Stimulates Anabolic Growth Responses in Explants of Articular Cartilage In Vitro

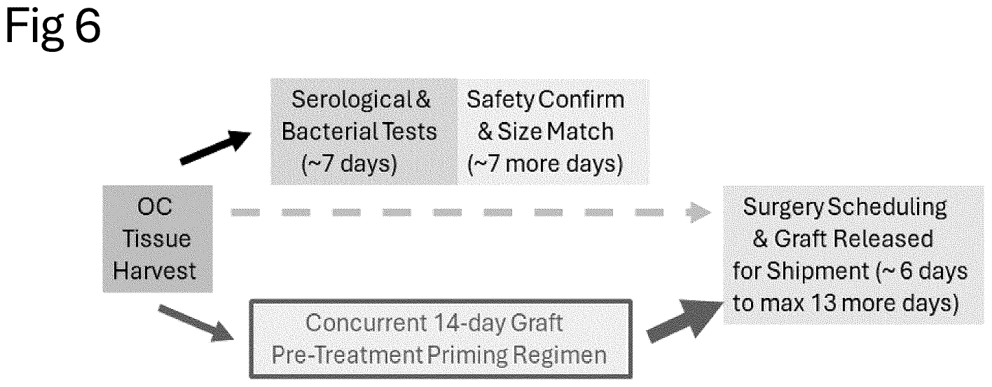

The standard tissue bank protocol for human osteochondral allograft storage and testing is diagrammed in FIG. 6. Following harvest, the tissue bank carries out FDA-mandated serological and bacterial screening tests which typically require about 14 days to complete. After completion, the graft is size matched to a recipient patient, the recipient surgeon is notified to schedule surgery, and the graft is shipped.

To accommodate this timeline, a 14-day pre-treatment regimen was developed in which HB-EGF (200 ng/ml) is intermittently added to the media of osteochondral explants in five alternating cycles of HB-EGF over that period (1-2 days HBEGF treated media alternated with 1-2 days control media).

This regimen was tested using explants of fresh adult goat femoral condylar articular cartilage. In this experiment, the response to HB-EGF by cartilage from the load bearing versus the non-load-bearing regions of the femoral condyle was compared. This is because the vast majority of clinical osteochondral defects occur in load bearing regions of the joint.

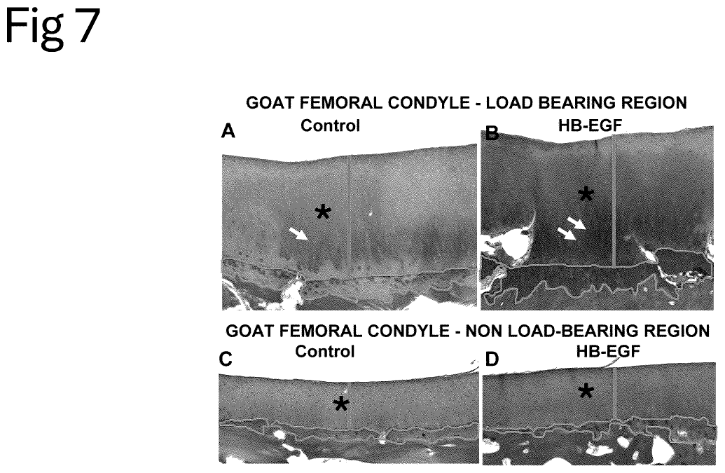

The load bearing and non-load bearing regions of the femoral condyle have distinct morphology as shown in FIG. 7 (FIG. 7A versus FIG. 7C) which are explants after 28 days in control media. In the osteochondral explant taken from the load-bearing region shown in FIG. 7A, the thickness of the noncalcified cartilage (vertical line) is ˜4 times the thickness of the calcified zone (outlined), while in the non-load-bearing explant shown in FIG. 7C, the noncalcified zone is only about twice the thickness of the calcified zone. Additionally, Safranin-O staining is markedly greater in load-bearing cartilage (compare intensity of gray shading in the cartilage mid-zone (asterisks) in FIG. 7A versus FIG. 7C). These characteristics indicate that articular cartilage from load-bearing regions contains relatively more aggrecan-rich hyaline cartilage matrix than non-load-bearing regions, reflecting the functional demands of load bearing.

Surprisingly, a difference in the HB-EGF response was observed between load bearing and non-load bearing cartilage. As shown in FIG. 7B, 14 days of five cycle HB-EGF promotes anabolic responses in load-bearing articular cartilage compared to non-load bearing cartilage FIG. 7A including markedly robust Safranin-O staining (compare intensity of gray shading in the midzone regions of FIG. 7B and FIG. 7A) and increased cell density (See, the arrow in FIG. 7B). This result is consistent with the anabolic responses to HB-EGF that we observed in human osteochondral explants (which are all taken from the load-bearing regions).

Surprisingly, as shown in FIG. 7 (FIG. 7C versus FIG. 7D), anabolic responses to the 14-day HB-EGF regimen were not observed in goat osteochondral explants taken from lateral, non-load-bearing regions of the femoral condyle. Indeed, as shown in FIG. 7C versus FIG. 7D, the lateral non-load-bearing regions of the femoral condylar cartilage, which are thinner than the load bearing regions and stain only weakly with Safranin-O, are comparable after 14 days whether they received control or five cycles of HB-EGF regimen (compare vertical lines and intensity of gray shading in the mid-zone (asterisk). This suggests that HB-EGF may preferentially promote anabolic responses in load-bearing articular cartilage, which may reflect the more hyaline nature of load bearing vs non-load-bearing articular cartilage.

Together, these results suggest that the 14-day pre-treatment HB-EGF priming regimen that has been developed offers a useful approach to stimulate anabolic responses by osteochondral allografts within a clinically compliant timeline and that is particularly relevant for the load-bearing osteochondral tissue used to provide donor osteochondral allografts in the clinic.

### Example 5—Mechanism of HBEGF Accelerating Short Term Growth Responses and Healing of Injured Cartilage In Vitro

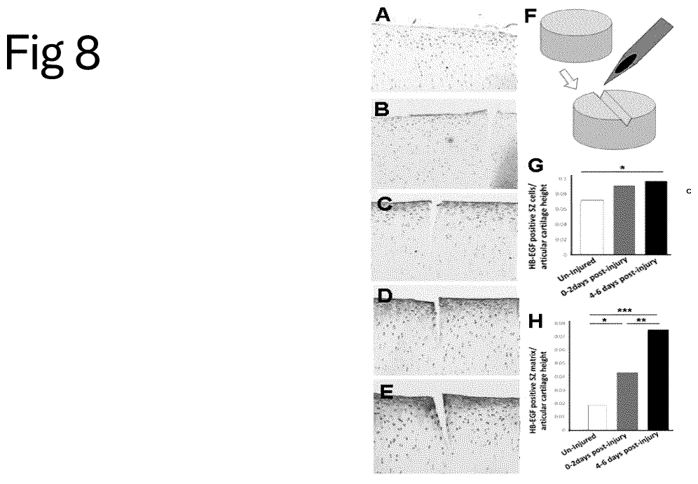

To examine short term responses by injured articular cartilage to HBEGF, articular cartilage explants were used that were either injured by scalpel scratch injury, or not injured, and followed over several days' time in vitro (no HBEGF treatment). FIG. 8 shows the injury model which is only as deep as the superficial zone, and is not a full thickness injury. FIGS. 8A-8E are immunostained sections showing localization of endogenous HBEGF in non-injured explants at the start of culture (See, FIG. 8A), and after injury (See, FIG. 7B=0-30 min, FIG. 7C=2 days, FIG. 7D=4 days; FIG. 7E=6 days). There is a rapid, prolonged increase in local HBEGF produced by superficial zone cells in response to superficial zone incisional wounding, which is a proliferative stimulus for superficial zone cells. This response is rapid, occurring in 30 minutes of injury. Thus, HB-EGF is a natural component of an early injury and healing response in cartilage.

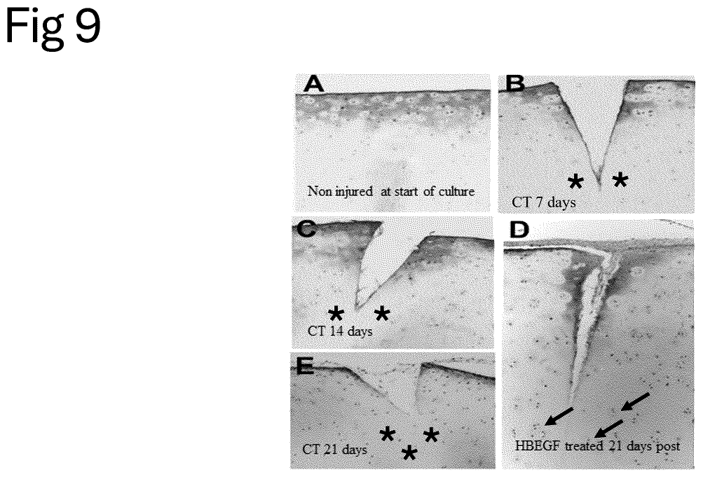

Explants that had received superficial zone injury were maintained for up to 3 weeks in vitro, with and without HBEGF treatment. FIGS. 9A-9D show sections of explants at various times immuno-stained with lubricin antibody, a SZ marker. At the start of culture (See, FIG. 7A), non-injured explants have abundant lubricin in the SZ but 7 days after injury (See, FIG. 7B), the cut is gaping and lubricin appears reduced, coincident with formation of a prominent dead zone around the cut where cells have died due to injury. Injured explants examined after 14 and 21 days are not different from those at 7 days except the dead zone is more extensive and loss of lubricin is more pronounced.

Remarkably however, if the injured explants were treated with HB-EGF (25-50 ng/ml) after injury and for 21 further days (See, FIG. 9D), HB-EGF promoted healing, as determined by narrower space between cut edges. Additionally, in the HB-EGF treated injured explants there is a reduced size of dead zone around the cut, abundant proliferating adjacent chondrocytes, and accompanying intense lubricin production. Lubricin immunostaining in the matrix of the SZ adjacent to the cut edges in the HB-EGF treated explant after 21 days was intermittently increased and decreased in a patchwork fashion along the cut edge, indicating simultaneous adjacent turnover and new matrix synthesis by the SZ progenitor cells at the cut edge. These results shed light into the mechanism whereby HB-EGF promotes anabolic articular cartilage responses including vitality, homeostasis, growth and injury healing.

### Example 6—Implanting an HB-EGF-Loaded Scaffold Matrix into a Critical Size Knee Articular Cartilage Injury in a Rabbit Model Induces a Cartilage Repair Response

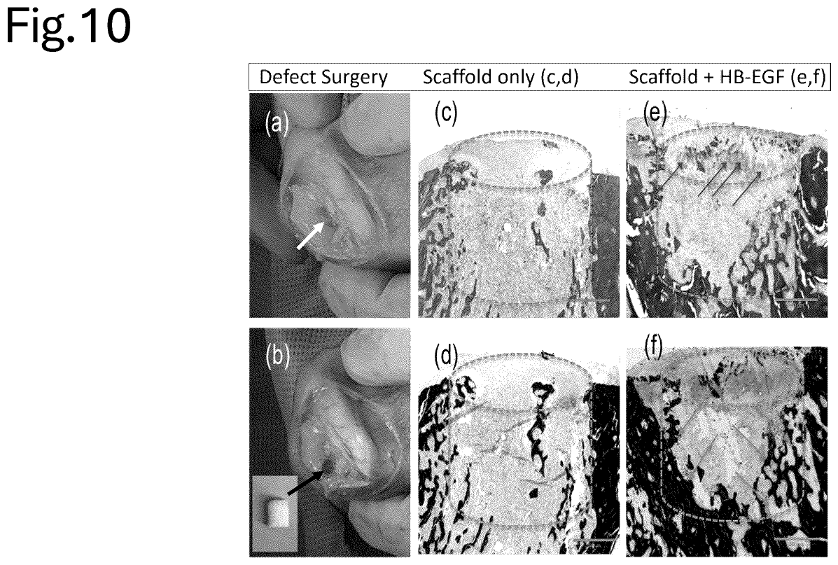

A pilot study was carried out to examine the ability of HB-EGF-loaded scaffolds to promote in vivo healing of articular cartilage injury. As shown in FIG. 10A and FIG. 10B, scaffolds with and without HB-EGF were implanted in a rabbit full-thickness knee osteochondral defect model. After 6 weeks, the tissue was harvested, and cartilage and bone formation evaluated histologically. In FIGS. 10C and 10D, osteo-chondral defects implanted with a scaffold alone (no growth factor) did not form any cartilage. In contrast, defects implanted with HB-EGF-loaded scaffold (See, FIGS. 10E and 10F) of the same type of material formed large areas of Safranin-O positive cartilage matrix that nearly bridged the upper portion of the defect (See, arrows in FIG. 10E). HB-EGF-loaded scaffolds were also well-integrated into the subchondral bone, as shown by Von Kossa-positive staining (See, arrows in FIG. 10F). These results validate the use of HBEGF as an agent that can be used as a coating or component of a synthetic or biological scaffold to enhance cartilage formation in surgical cartilage repair approaches for focal damage to the articular cartilage.

### Example 7—HBEGF is a Powerful Chemoattractant for Chondroprogenitor Cells

Unique features of HBEGF include its ability to regulate cohesion, an inherent mediator of the ability of a cell to change shape and assume migration, and its ability to bind to HSPG in the matrix, which increases bioavailability of HBEGF for its receptors on nearby cells, and could lead to establishment of invisible chemoattractant gradients of HBEGF that attract cells. The ability to migrate to locations of cartilage injury is a property of chondroprogenitors of the articular cartilage and may be involved in cartilage healing. The ability of HBEGF to act as a chemoattractant for SZ cells was investigated. The approach used an available chemotaxis assay system that reproduces 3D matrix and stable gradients used by mesenchymal cells during migration, and which critically provides information about nonspecific chemotactic responses (chemokinetic) which are purely random motions. HBEGF was compared to SDF1a, a factor that is a chemoattractant for immune cells that don't use mesenchymal chemotaxis movement.

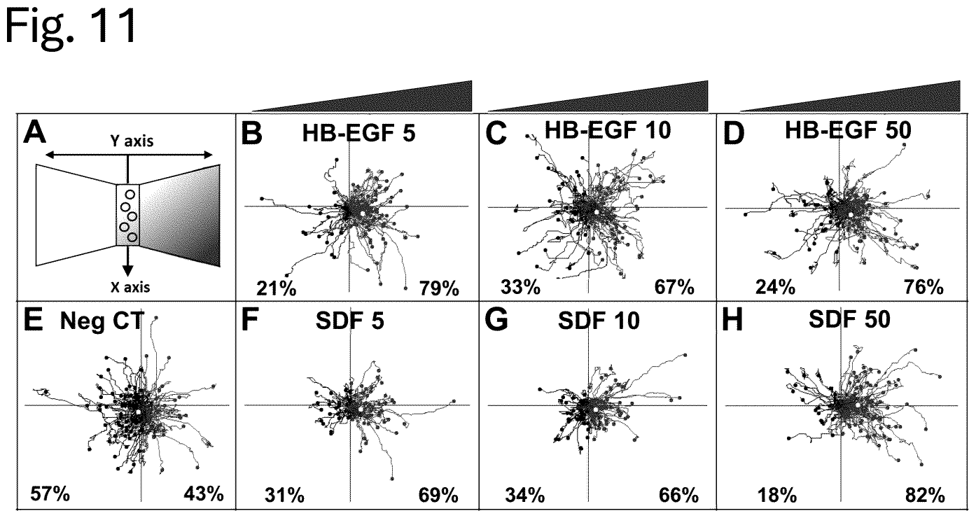

As shown in FIG. 11, trajectories of SZ cells over 24 hours were tracked in the assay system diagrammed in (See, FIG. 11A). In the absence of a gradient (See, FIG. 11E) there is no preferential directed migration. In the presence of gradients of HB-EGF (See, FIG. 11B-FIG. 11D) or SDF1a (See, FIG. 11F-FIG. 11H, there are 2-4 times more trajectories moving towards the gradients (gray tracks, p<0.05) in response to gradients of 5 ng/ml HB-EGF (See, FIG. 11B) and 50 ng/ml SDF-1α (FIG. 11H). However, the Center of Mass which is the average movement of all cells in each group (represented by the white dot) is displaced towards the growth factor gradient only in the 5 ng/ml HB-EGF group (See, FIG. 11C), indicating true chemotaxis response by the population of cells.

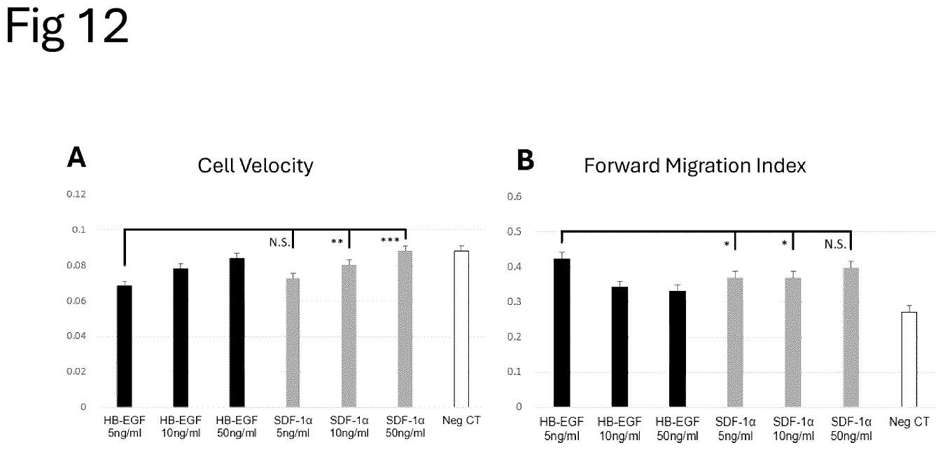

Further, as shown in FIG. 12, the average SZ cell velocity (see, FIG. 12A) and Forward Migration Index (B) are responsive to HB-EGF induced chemotaxis. In FIG. 12A, cell velocity±SD is shown in response to gradients of 5, 10, or 50 ng/ml HB-EGF (black bars) or SDF-1a (gray bars); or no growth factor (negative control, open bar). Velocity is slowest in response to a gradient of 5 ng/ml HB-EGF; velocity is faster in response to a gradient of 5 ng/ml SDF1a but this difference is not significant (ns). Velocities in response to HB-EGF gradients at 10 and 50 ng/ml are significantly slower than in response to SDF-1a gradients at the same concentrations (**p<0.01; ***p<0.001). Velocity in the absence of any gradient reflects random movement (chemokinesis). This data shows that chemokinesis occurs at 50 ng/ml.

FIG. 12 (B) shows the values for Forward Migration Index (FMI) for SZ cell movement over a 24-hour period in the presence of 5, 10, or 50 ng/ml HB-EGF (black bars) or SDF-1a (gray bars); or no growth factor (negative control, open bar). FMI-y is greatest in response to lowest concentration of HB-EGF (5 ng/ml); FMI-y values are significantly lower in other groups except the highest concentration of SDF1a (50 ng/ml, ns) which is chemokinetic (*p<0.05).

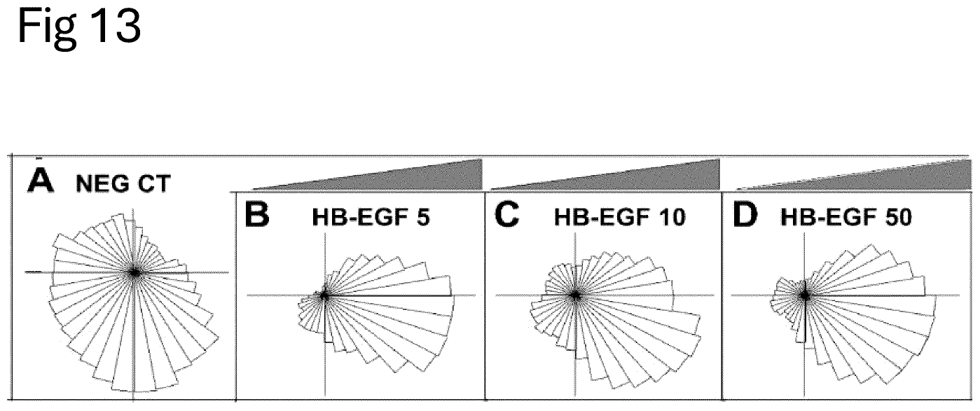

The histogram plots in FIG. 13 depict the relative frequencies of trajectories taken by SZ cells over a 24-hour period in the absence of a gradient (See, FIG. 13A) or in the presence of gradients of 5, 10, or 50 ng/ml HB-EGF (See, FIGS. 13B-13D). In the presence of 5, 10, or 50 ng/ml HB-EGF (See, FIGS. 13B-13D), most migration angles are clustered among directional quadrants representing forward or parallel movement in relation to the concentration gradient. In the absence of growth factor (See, FIG. 13A, Negative Control), most migration angles are clustered in quadrants representing opposite or perpendicular movement. Note that only in the presence of 5 ng/ml HBEGF (See, FIG. 13B) are there no migration angles opposite to the concentration gradient (*).

Together these results demonstrate that SZ cells are exquisitely sensitive to HBEGF as a chemoattractant, and HBEGF was a superior chemoattractant for SZ cells at concentrations lower than used for SDF1a. HBEGF regulates cohesion and migration through HER4 signaling especially surface signaling via pro-ligand form, and its binding to matrix heparan sulfate proteoglycans gives it the ability to form nano or microscale concentrations gradients. It is suggested that these properties (proligand signaling, HER4 activation, and HSPG binding) are critical characteristics of HBEGF that make it unique as an agent for stimulation of progenitor injury responses that can be leveraged towards clinical cartilage repair and regeneration. For example, HBEGF could be useful as a coating on a biological or synthetic or processed graft, or as a component of implanted cells, that could recruit native cells to the implant site to improve healing and sustain or extent graft life.

### Example 8—HBEGF Promotes Chondrogenic Differentiation In Vitro

The effects of HBEGF on stimulation of chondroprogenitor differentiation in vitro were examined in micromass high density cultures, a system in which spontaneous differentiation of limb mesenchymal cells into the cartilage lineage occurs.

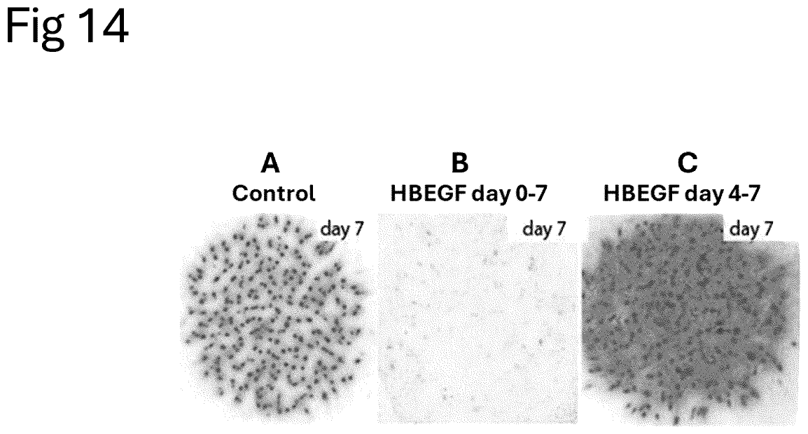

As shown in FIG. 14A, mesenchymal cells differentiate into cartilage nodules after 7 days as determined by their synthesis of Alcian blue stained matrix. As shown in FIG. 14B, when provided to mesenchymal cells at the start of culture, and examined 7 days later, chondrogenic differentiation was nearly completely impaired by HB-EGF. However, as shown in FIG. 14C, surprisingly, when provided after the cells had already entered the cartilage lineage, which occurs around day 4, chondrogenic differentiation at day 7 was markedly promoted by HBEGF, as measured by size of the micromass diameter, abundance of cartilage nodules and its nearly uniform intensity of Alcian blue staining, a readout of new cartilage matrix synthesis. This suggests that HBEGF signaling is involved in chondroprogenitor expansion responses and or cartilage phenotypic maintenance. HBEGF regulates cohesion through its unique pro-ligand form; HBEGF also binds heparan sulfate proteoglycans in the matrix. Since cohesion and HSPG binding are critical components of cartilage phenotypic maintenance, these results provide support for utility of HBEGF as a component of procedures for promoting chondroprogenitor expansion in cartilage grafts, and as a factor that promotes healing by natural or synthetic grafts. Moreover, these results provide direct support for utility of HBEGF as an agent that promotes chondrocyte expansion or chondro-progenitor expansion required for MACI or other cell-based procedures.

Embodiments disclosed here are not limiting of the subject matter and is merely exemplary. Various other components may be included and called upon for providing for aspects of the teachings herein. For example, additional materials, combinations of materials and/or omission of materials may be used to provide for added embodiments that are within the scope of the teachings herein.

The following terms are used to describe the invention of the present disclosure. In instances where a term is not specifically defined herein, that term is given an art-recognized meaning by those of ordinary skill applying that term in context to its use in describing the present disclosure.

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

Compounds and materials are described using standard nomenclature. Unless defined otherwise, all technical and scientific terms used herein have the same meaning as is commonly understood by one of skill in the art to which this invention belongs. The following terms are used to describe the invention of the present disclosure. In instances where a term is not specifically defined herein, that term is given an art-recognized meaning by those of ordinary skill applying that term in context to its use in describing the present disclosure.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

As used herein, the term “substantially” means to a great or significant extent, but not completely.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

The terms “comprising”, “having”, “including”, and “containing” are to be construed as open-ended terms (i.e., meaning “including, but not limited to”) unless otherwise noted.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. All ranges disclosed herein include both end points as discrete values as well as all integers and fractions specified within the range. For example, a range of 0.1-2.0 includes 0.1, 0.2, 0.3, 0.4 . . . 2.0. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, the term “administering” means the actual physical introduction of a composition into or onto (as appropriate) a subject, a host, or cell. Any and all methods of introducing the composition into the subject, host or cell are contemplated according to the invention; the method is not dependent on any particular means of introduction and is not to be so construed. Means of introduction are well-known to those skilled in the art, and also are exemplified herein. “Providing” means giving, administering, selling, distributing, transferring (for profit or not), manufacturing, compounding, or dispensing.

As used herein the term “bone morphogenetic proteins” (BMPs) or “bone morphogenetic protein family member”, as used interchangeably herein, refers to proteins that belong to the transforming growth factor (TGF) B-superfamily and play critical roles in the development, growth, cell differentiation and cartilage and bone morphogenesis. Examples of BMP: include BMP-2, BMP-3, BMP-3B, BMP-4, BMP-5, BMP-6, BMP-7, BMP-8, BMP-8B, BMP-9, BMP-10, BMP-12, BMP-13, BMP-14, BMP-15, or any combination thereof.

As used herein, the phrase, “heparan-binding epidermal growth factor (EGF)-like growth factor”, “heparin-binding epidermal growth factor”, or “HB-EGF” as used interchangeably herein, refers to a member of the EGF superfamily and is expressed by many cell types and is produced in multiple tissues such as lung, skeletal muscle, brain and heart. HB-EGF participates in a variety of physiological and pathological processes including development, wound healing, blastocyst implantation, atherosclerosis and tumor formation. The human EB-EGF amino acid sequence can be found at Uniprot Q99075.

As used herein, the phrase “natural growth factor” refers to substances (e.g., a protein, hormone, a nucleic acid, or combinations thereof) capable of stimulating one or more of cell proliferation, wound healing, and/or cellular differentiation. Growth factors are generally important in regulating a variety of cellular processes. Examples of natural growth factors include heparin-binding epidermal growth factor (HB-EGF), epidermal growth factor (EGF), transforming growth factor alpha (TGF-α), amphiregulin (AREG), betacellulin (BTC), epiregulin (EREG), epigen, neuregulin 1-4 (NRG1-4) and isoforms thereof, and any combinations thereof. In some embodiments, the natural growth factor is HB-EGF. In other embodiments, the natural growth factor is EGF. In other embodiments, the natural growth factor is TGF-α. In other embodiments, the natural growth factor is AREG. In other embodiments, the natural growth factor is BTC. In other embodiments, the natural growth factor is EREG. In other embodiments, the natural growth factor is epigen. In other embodiments, the natural growth factor is neuregulin 1-4 and isoforms thereof.

As used herein, “optional” or “optionally” means that the subsequently described event or circumstance may or may not occur, and that the description includes instances where said event or circumstance occurs and instances where it does not.

The term “subject” or “patient”, as used interchangeably herein, refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject or patient is a human subject or a human patient, such as a human being treated or assessed for a disease, disorder or condition, a human at risk for a disease, disorder or condition, a human having a disease, disorder or condition, and/or human being treated for a disease, disorder or condition as described herein. In one embodiment, the subject is about 1, 2, 3, 4, 5, 6, 7, 8, 9, or 10 years of age. In another embodiment, the subject is about 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100 years of age. Values and ranges intermediate to the above recited ranges are also intended to be part of this invention. In addition, ranges of values using a combination of any of the above-recited values as upper and/or lower limits are intended to be included. As used herein, a subject is “in need of treatment” if such subject would benefit biologically, medically, or in quality of life from such treatment. A subject in need of treatment does not necessarily present symptoms, particular in the case of preventative or prophylaxis treatments.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and ¹³¹I.

A significant change is any detectable change that is statistically significant in a standard parametric test of statistical significance such as Student's t-test, where p<0.05.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112(f) interpretations as “means-plus-function” language unless specifically expressed as such by use of the words “means for” or “steps for” within the respective claim.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

### Other Embodiments

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method for restoring, repairing, or regenerating cartilage in a mammal in need thereof, the method comprising the step of: injecting or implanting in to a joint of a mammal a composition, wherein the composition comprises at least one of (a) a tissue; (b) a natural or synthetic scaffold, or combination thereof; (c) a hydrogel; (d) a membrane; (e) chondrocytes or chondrogenic cells; (f) a particle; or (g) any combination of (a)-(f), and further wherein any of (a)-(g) of the composition have been treated with or exposed to at least one natural growth factor prior to injection or implantation into the joint of the mammal.

2. The method of claim 1, wherein the natural growth factor is heparin-binding epidermal growth factor (HB-EGF), epidermal growth factor (EGF), transforming growth factor alpha (TGF-α), amphiregulin (AREG), betacellulin (BTC), epiregulin (EREG), epigen, neuregulin 1-4 (NRG1-4) and isoforms thereof, or a bone morphogenetic protein (BMP).

3. The method of claim 1, wherein the mammal is a human, pig, dog, cat, cow, horse, guinea pig, goat, sheep, mouse, or horse.

4. The method of claim 1, wherein the mammal suffers from Osteochondritis Dissecans or osteoarthritis.

5. The method of claim 1, wherein the composition comprises a natural or synthetic scaffold or combination thereof and further wherein the (a) natural scaffold comprises, collagen, aragonite, gelatin, elastin, hyaluronic acid, chitosan, chondroitin sulfate, agarose, alginate, cellulose, fibrin, or any combinations; thereof; and (b) synthetic scaffold comprises poly(ethylene glycol) (PEG), polycaprolactone (PCL), polylactic acid (PLA), polyurethane (PU), poly(glycolic acid) (PGA), polyethersulfone (PES), poly-p-dioxanone (PDS), poly(l-lactide) (PLLA), polylactic-co-glycolic acid (PLGA), polycaprolactone-polyethylene glycol (PCEC), polysulfone, or any combinations thereof.

6. The method of claim 1, wherein the composition comprises tissue and the tissue is osteochondral tissue, chondral tissue, or a combination thereof.

7. The method of claim 6, wherein the osteochondral tissue, chondral tissue or combination thereof is an allograft or an autograft.

8. The method of claim 1, wherein the composition comprises a particle and the particle is a microsphere.

9. The method of claim 1, wherein the joint is a knee, a hip, a shoulder, an elbow, a wrist, an ankle, a digit, a spine, a temporomandibular joint, or any combination thereof.

10. The method of claim 1, wherein the composition further comprises heparinase, heparan sulfate, or a combination thereof.

11. The method of claim 2, wherein the composition comprises at least one bone morphogenetic protein.

12. The method of claim 11, wherein the bone morphogenetic protein is BMP2, BMP3, BMP3B, BMP4, BMP5, BMP6, BMP7, BMP8, BMP8B, BMP9, BMP10, BMP12, BMP13, BMP14, BMP15 or any combination thereof.

13. A method of increasing the shelf-life of a composition comprising an osteochondral or chondral allograft or osteochondral or chondral autograft, the method comprising, adding at least one heparin-Binding Epidermal Growth Factor (HB-EGF), a bone morphogenetic protein, or a combination thereof, to a composition comprising an osteochondral or chondral allograft or osteochondral or chondral autograft.

14. The method of claim 13, wherein the shelf-life of the composition comprising the HB-EGF is increased at least one week when compared to a composition comprising an osteochondral or chondral allograft or osteochondral or chondral autograft that does not contain at least one HB-EGF.

15. The method of claim 13, wherein the method further comprises adding a particle comprising at least one natural growth factor to the composition.

16. The method of claim 15, wherein the at least one natural growth factor is HB-EGF, BMP2, BMP9 or any combinations thereof.

17. The method of claim 16, wherein the particle is a microsphere.

18. The method of claim 17, wherein the HB-EGF is coated on the microsphere or embedded in the microsphere.

19. The method of claim 13, wherein the composition further comprises heparinase, heparan sulfate, or a combination thereof.
